(19) United States
(12) Patent Application Publication
(10) Pub. No.: US 2005/0106594 A1
(43) Pub. Date: May 19, 2005

(54) In vitro selection of aptamer beacons

(76) Inventors: Andrew Ellington, Austin, TX (US); Manjula Rajendran, Redwood city, CA (US)

Correspondence Address: David L. Parker;FULBRIGHT & JAWORSKI L.L.P., 600 Congress Avenue, Suite 2400, Austin, TX, 78701, US

(21) Appl. No.: 10/924,144

(22) Filed: Aug. 23, 2004

Related U.S. Application Data
(60) Provisional application No. 60/497,104, filed on Aug. 22, 2003.

(51) Int. Cl. C12Q 1/68; C07H 21/04; C12P 19/34

(52) U.S. Cl. 435/6; 435/91.2; 536/25.32

(57) ABSTRACT

Provided herein are methods of selecting aptamer beacons in vitro using single-stranded nucleic acid species comprising a fluorphore and a random region of N nucleotides. The single-stranded nucleic acid species are annealed to a capture oligonucleotide comprising an F1 quenching moiety. Aptamer beacons are eluted using a target ligand or analyte to interact with the captured single-stranded nucleic acid species to release it from the capture oligonucleotide. Those selected single-stranded nucleic acid species comprise aptamer beacons. Also provided are the aptamer beacons so selected and methods of detecting a ligand in solution using selected aptamer beacons.

### CROSS-REFERENCE TO RELATED APPLICATIONS

This nonprovisional applications claims benefit of provision U.S. Ser. No. 60/497,104, filed Aug. 29, 2003, now abandoned.

### BACKGROUND OF THE INVENTION

1. Field of the Invention

The present invention relates generally to the fields of biochemistry, nucleic acid chemistry and fluorescence spectroscopy. More specifically, the present invention relates to in vitro selection of molecular beacons.

2. Description of the Related Art

Molecular beacons are oligonucleotide probes that assume a hairpin structure in which the single-stranded loop can pair with complementary sequences and the paired stem contains fluorescent reporters (or a fluorphore and a quencher) that interact with one another (1). Hybridization of a complementary target sequence leads to the formation of a long duplex region, destabilization of the hairpin, and a spatial separation between the two dyes. Ultimately, interaction with target oligonucleotides leads to either the loss of fluorescence resonance energy transfer (FRET) or to dequenching of a fluorphore, optical signals that can be readily detected. An alternate form of molecular beacons called tripartite molecular beacons have been engineered (2). The hairpin-stem of the tripartite molecular beacon does not have the fluor and quencher directly attached to it, but rather is extended by two universal single-stranded arms that bind single-stranded oligonucleotides having fluorphore or quencher attached to them.

The simplicity of the transduction mechanism and the corresponding ease with which molecular beacons can be designed has led to their adoption in a variety of applications, including identifying single nucleotide polymorphisms (3-5), detecting pathogens (6-7), monitoring the amplification of nucleic acids during real-time PCR (8-9), and detecting DNA-RNA hybridization in real-time in living cells (10-11). Immobilized molecular beacons have also been used to detect DNA-RNA hybridization (12-13), including in DNA arrays (14). The oligonucleotide-dependent conformational changes that characterize molecular beacons have also been engineered into deoxyribozymes, yielding oligonucleotide-dependent changes in catalytic activity, or so-called catalytic molecular beacons (15-16).

Molecular beacons have primarily been designed to recognize oligonucleotides. However, while molecular beacons can detect nucleic acid targets with high specificity and with single mismatch discrimination, their ability to function as biosensors for the detection of analytes other than nucleic acids has so far been relatively limited. Molecular beacons have been used to probe the interactions of known nucleic acid binding proteins with single-stranded DNA; for example, single-stranded DNA binding proteins can open a molecular beacon as well or better than a complementary target (17-19). However, these approaches have not proven to be generalizable. Sensors similar to molecular beacons have also been adapted to the detection of proteins that interact with double-stranded DNA targets (19). In this instance, the DNA binding protein assembles two sub-fragments that contain different dyes, leading to a fluorescence resonance energy transfer (FRET) signal.

In comparison, it has proven possible to select nucleic acid binding species (aptamers) that can bind to a wide variety of targets, from small organic molecules to large supramolecular structures (20-22). Aptamers can potentially be adapted to function as nucleic acid biosensors in a variety of ways (23-24). For example, aptamers frequently undergo small ligand-induced conformational changes. When fluorescent labels were introduced into conformationally labile positions in anti-adenosine aptamers, the resultant ‘signaling aptamers’ showed ATP-dependent increases in fluorescence intensity and could track the concentration of free ATP in solution (25). Larger conformational changes can also be exploited. An anti-thrombin DNA aptamer was known to assume an equilibrium between random coil and quadruplex structures. By labeling the aptamer with either a fluorphore and a quencher or two fluorphores (26) addition of thrombin shifted the equilibrium to the quadraplex conformer and resulted in fluorescence-quenching or FRET.

In addition to exploiting the inherent conformational changes that aptamers undergo, aptamers that are similar to molecular beacons have been generated by engineering the aptamer such that the addition of an analyte resulted in a large conformational change and concomitant diminution or increase in a fluorescent signal. In a strategy similar to that described above for the thrombin biosensor, an anti-cocaine aptamer was mutated and its secondary structure destabilized; the addition of cocaine resulted in stem formation and fluorescence-quenching (27). The strategies that have so far been described have generally resulted in quenching, which could occur by a variety of mechanisms. In contrast, the addition of sequences to the anti-thrombin DNA quadruplex forced the adoption of an alternate, hairpin conformation, similar to the hairpin or ‘closed’ conformation of a molecular beacon. Upon addition of thrombin, the aptamer resumed its quadruplex conformation, splitting apart an appended fluorphore and quencher and yielding a substantial fluorescent signal (28).

Finally, even quaternary structural changes have been exploited to create aptamer biosensors. An anti-Tat aptamer was split into two pieces, one of the pieces was converted into a molecular beacon, and the Tat-dependent reassembly of the aptamer resulted in the opening of the beacon and the generation of a fluorescent signal (29). Anti-cocaine and anti-rATP aptamers have also been converted into fluorescent sensors for their respective analytes using a similar strategy of target-mediated assembly, although in this instance analyte-binding leads to hairpin stem formation and hence to fluorescence quenching (30). Recently, a similar strategy was employed to convert anti-ATP and anti-thrombin aptamers into signaling aptamers. In this case, an antisense oligonucleotide bound to, denatured, and quenched a fluorescently-labeled aptamer. Target-binding stabilized the native conformation of the aptamer and resulted in fluorescence dequenching (31).

However, all of these methods ultimately rely upon engineering known aptamers to generate signals and frequently require a prior knowledge of the detailed secondary or tertiary structure of the aptamer. Even when these details are known, multiple, different constructs must frequently be generated in order to identify those that signal or that signal most efficiently. It would be simpler to directly couple selection with signaling. Selected aptamers from pools that randomly incorporated fluorescent nucleotide derivatives and that therefore pre-positioned fluorphores into functional nucleic acid structures have been employed (32). However, since the selected pool could still only be selected for binding, rather than for signaling, the selected aptamers had to be individually screened to identify those that could not only bind but also could signal. Not all high-affinity binding species are also good at signal transduction; only one family of aptamers was found to have significant signaling abilities.

Metal ions are known to interact both specifically and non-specifically with nucleic acids and should make good targets for selection. In fact, all nucleic acid biopolymers require metal ions for proper folding and maintenance of their tertiary structure and also for function (33) Sensors for metal ions which enable their specific, sensitive, and real time detection are useful and important in a variety of applications ranging from environmental monitoring, and clinical diagnostics or toxicology studies, to in vivo studies for elucidating their roles in biology. Numerous sensors have been described based on small organic molecules, peptides, proteins, or even whole cells as receptor components. Among the different metal ions, considerable progress has been made in the development of sensors against biologically important metal ions such as Ca²⁺ (34-38) and Zn²⁺ ions (39-43).

In vitro selection has been employed previously to select Zn²⁺ binding RNA aptamers using a Zn²⁺ affinity column. The RNA aptamers were however not very sensitive; while the originally selected aptamers had a K_{d} of ˜1 mM, re-selection gave RNA aptamers having a binding affinity of ˜100-400 mM (44-45). The selected aptamers could also recognize a variety of metal ions. Similarly, in vitro selection has also been employed to isolate novel nucleic acid enzymes which require a specific metal ion for activity, or to change the metal ion specificity of existing enzymes. For example, Pan and Uhlenbeck selected Pb²⁺-dependent ribozymes, (46) while Breaker and Joyce selected Pb²⁺-dependent RNA cleaving deoxyribozymes (47). In vitro selection was also used to evolve the group I intron (48) and the RNase P ribozymes (49) to utilize Ca²⁺ instead of Mg²⁺. In the same way, Cu²⁺ dependent deoxyribozymes (50-52) and Zn²⁺ dependent RNA cleaving deoxyribozymes (53-54) have also been selected. In particular, the deoxyribozyme identified from the Zn²⁺ selection by Lu's group showed a better responsivity for Pb²⁺ ions. This deoxyribozyme has thereafter been converted into a sensitive fluorescent and colorimetric sensor for Pb²⁺ ions (55-57) Nonetheless, the development of sensitive and selective metal ions sensors which report only the target metal ion even in the presence of other interfering metals, remains a major challenge.

Similarly, the Zinpyr-1, Zinpyr-2 and ZP4 sensors show a 3- to 5- fold fluorescence enhancement on binding Zn²⁺(39,58). This latter sensor however has much better binding affinities for zinc when compared to the selected aptamer sensor. In general, these organic macrocylic receptors are able to bind zinc with affinities comparable to that of natural zinc receptor, such as carbonic anhydrase, by coordinating the zinc ion via multiple nitrogen and oxygen ligands. In Zinpyr-1, for instance, the Zn2+ ion is coordinated in a trigonal bypyramidal geometry by 3 nitrogen atoms of the receptor, di-(2-picolyl)amine or DPA, arm, an oxygen atom of a henolic group and a water molecule. In the case of the selected aptamer, it is possible that the aptamer is not able to displace all the water molecules froming the hydration sphere around the metal ion and is hence not able to achieve such tight binding.

Ideally, it would be extremely useful if a generalizable strategy could be employed to develop sensors against a variety of different metal ions. However, once a sensor has been developed, it is often very challenging to change its selectivity, both with chemically designed and biological receptors. Zinc is an essential transition metal element that is indispensable for growth and development and known to participate in diverse biological processes ranging from RNA synthesis, and regulation of gene expression, to metabolism, apoptosis and neuronal signaling in the cortex of the brain (59).

The most well known biological function of zinc is its role as a structural and catalytic component of proteins. In addition, while most of the zinc in biological systems is tightly bound within proteins, free or loosely bound zinc is present in high concentrations in the brain and many specialized secretory vesicles including in the pancreas, pituitary, prostate and leucocytes (41). Of particular interest in the recent years has been the putative role of zinc as a signaling ion in the nervous system (60-61). In addition to its role as a neurotransmitter, zinc ions have also been implicated in the pathology of several neurodegenerative diseases (62).

However, despite the significance of zinc in biological systems, not much is known about the details of Zn²⁺ homeostasis. Zinc levels must be well regulated in healthy cells since high concentration of free zinc is known to be toxic to cells. Although several Zn²⁺ transport proteins and metallothioneins are known, (63-64) many aspects of zinc uptake, its transport, distribution and incorporation into proteins and enzymes in the required amounts, and the regulation of these processes are not well understood. The low intracellular concentrations of zinc, and the fact that it is spectroscopically silent, have made the study of this metal ion very difficult.

To analyze the in vivo roles of zinc, numerous fluorescent zinc indicators, (40-41) based on peptides, proteins (for example, those based on carbonic anhydrase (43) and zinc finger motifs (42,65) and organic macrocyclic receptors (for example, TSQ, Zinquins, ZnAFs, (66-68) and Zinpyrs (38-39,58) have been developed. The major challenges in developing such sensors for zinc are specificity and sensitivity. Although in specialized cells which are abundant in Zn²⁺, the Zn²⁺ concentration can be as high as low millimolar, the typical concentration within the cytosol of normal cells is low nanomolar or picomolar (41). At the same time, other divalent metal ions like magnesium and calcium are present in high concentrations (mM- mM) and may interfere with Zn²⁺ determination.

The generality of extending molecular beacon design to other types of analytes is currently unclear. Thus, in order to generalize the utility of molecular beacons to analyte classes other than nucleic acids, the inventors have recognized a continuing need for a method for the direct selection of signaling aptamers. The prior art is deficient in the lack of in vitro selection methods for aptamer beacons. Specifically, the prior art lacks in vitro selection methods of aptamer beacons that directly couple selection for analyte- or ligand-binding to a nucleic acid conformational change that produces a fluorescent signal. The present invention fulfills this long-standing need and desire in the art.

### SUMMARY OF THE INVENTION

The present invention is directed to, inter alia, a method of selecting aptamer beacons in vitro. A pool of single-stranded nucleic acid species is generated which comprises a fluorphore F1 and a random insert of N nucleotides. The F1-labeled single-stranded nucleic acid species is annealed with a capture oligonucleotide to form a capture pool where the capture oligonucleotide comprises F1 quenching moiety Q1. The capture pool is immobilized on a column and eluted with at least one target. An eluate comprising the F1-labeled single-stranded nucleic acid species is amplified. These method steps may be repeated to select for the F1-labeled single-stranded nucleic acid species which comprise aptamer beacons.

The present invention also is directed to a method of selecting a family of aptamer beacons in vitro using a ssDNA as the nucleic acid species. The aptamer beacons are selected and cloned as described. Additionally, the clones are sequenced where clones having a motif comprising common residues at or near the 5′ end of the random insert comprise a family of aptamer beacons.

The present invention is directed further to a method of detecting a ligand in solution using the aptamer beacons selected by the method described herein. An initial level of fluorescence of a fluorphore F1 attached within the 5′ region of an aptamer beacon described supra is determined and the F1- molecular beacon is annealed with a capture oligonucleotide that comprisies an F1 quenching moiety to form a captured beacon construct. The captured beacon construct is immobilized and contacted with the solution. The ligand interacts with the captured beacon whereby the captured beacon is released from the capture oligonucleotide and an increase in fluorescence of F1 from the quenched state of F1 is determined upon the release of the captured beacon thereby detecting the ligand.

The present invention is directed to a related method of detecting a ligand in solution using an aptamer beacon comprising an additional fluorphore F2 different from the fluorphore F1 and which exhibits a fluorescent color distinct from F1. F2 is attached within the 5′ region of the random insert of the aptamer beacons described herein. The aptamer beacon further comprises a 5′ endl-inked fluorescence quenching moiety on the 5′ region of the aptamer beacons. When captured by the capture oligonucleotide, F1 is quenched and F2 fluoresces. In the presence of ligand, the interaction thereof releases the molecular beacon such that Q2 quenches F2 and F1 fluoresces. The change in fluorescent color upon addition of solution detects the presence of ligand in the solution.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention are briefly summarized. The above may be better understood by reference to certain embodiments thereof which are illustrated in the appended drawings. These drawings form a part of the specification. It is to be noted; however, that the appended drawings illustrate preferred embodiments of the invention and therefore are not to be considered limiting in their scope.

FIGS. 1A-1B depict the selection scheme for molecular beacons. FIG. 1A shows the conformational changes in designed molecular beacons. F represents an embedded fluorphoree, Q a quencher. FIG. 1B shows the in vitro selection of molecular beacons. The closed circle at the termini of the capture oligonucleotide represents biotin.

FIGS. 2A-2D depict the effect of oligonucleotide length on affinity column retention. FIG. 2A shows the capture oligonucleotide was designed to be complementary to the 5′ end of the pool. Symbols are as in FIGS. 1A-1B. FIG. 2B is the comparison of four different capture oligonucleotides. W1 through W9 indicate fractions obtained after washing the column with one column volume of selection buffer. FIG. 2C shows the residual retention of the pool on the column. To determine the extent of elution with the 12-mer and 15-mer capture oligonucleotides the experiments described in FIG. 2B were repeated, except that denaturing buffer containing 7M urea was used. Fractions are the same as in FIG. 2B. FIG. 2D shows the oligonucleotide sequences adopted for selection experiments from the N20 and N50 pools. ‘F’ indicates Fluorescein, while ‘Q’ indicates a pendant DABCYL.

FIGS. 3A-3D depict the progress of the selection. FIG. 3A shows the binding assays with Round 9 beacons. The horizontal axis indicates the percents of the pools or beacons that were specifically eluted from an oligonucleotide affinity column. Yellow bars show elution with a mixture of oligonucleotide targets OT1 and OT2, while the blue bars show elution with oligonucleotide target OT2 alone. FIG. 3B shows the sequences of Round 9 beacons. The constant primer-binding regions are shown in lowercase grey while the random region has been shown in uppercase bold. The common octamer motif is shown in bold red. FIG. 3C shows the beacon sequence complementarity. Predicted base-pairings to OT2 are shown. A stem that is predicted to form between the constant region and the common octamer (red) is shown underlined. FIG. 3D shows the specificity of elution. The total amount of beacons 14a and 16c that were eluted from an oligonucleotide affinity column with an equimolar amount of the oligonucleotide targets, OT2, OT1, and T21 is shown.

FIGS. 4A-4B depict the progress of selection from the N50 pool. FIG. 4A shows the selection/amplification conditions for each of 12 rounds of selection from the target pool. FIG. 4B shows the target dependent elution after each round of selection.

FIGS. 5A-5B depicts the sequences of the thirty four aptamers selected from the N50 pool. FIG. 5A shows the primary amino acid sequence of Family Zn1 and Family Zn2 aptamers. FIG. 5B shows the secondary loop structure formed by aptamers.

FIGS. 6A-6C depict the mechanism of elution for beacon 14a. FIG. 6A shows a proposed mechanism of elution. Representations are as in FIG. 3C. Hybridization of the oligonucleotide target OT2 stabilizes the formation of a hairpin stem and disrupts interactions with the capture oligonucleotide. FIG. 6B demonstrates assaying the mechanism of elution with beacon variants. Beacons designed to assess interactions with OT2 or the formation of the intramolecular hairpin (underlined) are shown. The elution characteristics of these constructs with target OT2 were assessed as in FIG. 3A. FIG. 6C demonstrates assaying the mechanism of elution with target oligonucleotide variants. Targets designed to assess interactions between OT2 and beacon 14a are shown. The elution characteristics of beacon 14a with the target variants were assessed as in FIG. 3A.

FIGS. 7A-7B depict the mechanism of elution for beacon 16c. FIG. 7A shows the proposed mechanism of elution. Representations are as in FIG. 3C. FIG. 7B demonstrates assaying the mechanism of elution. Beacons and target oligonucleotides designed to assess the mechanism of elution are shown. The elution characteristics of beacon 16c were assessed as in FIG. 3A.

FIGS. 8A-8C show designed molecular beacons. FIG. 8A is a designed molecular beacon based on the proposed elution mechanism for beacon 14a. Two molecular beacons cOT1 and cOT3 and three target oligonucleotides OT1, OT3, and OT3b were designed. FIG. 8B depicts the proposed role of a hypothesized, immobilized secondary structure in the mechanism of elution. The hypothesized secondary structures of the selected and designed beacons are shown. FIG. 8C demonstrates the elution characteristics of molecular beacons cOT1 and cOT3 with targets OT1, OT3, and OT3b were assessed as in FIG. 3A. The representations are as in FIG. 3C.

FIGS. 9A-9J depict beacon variants for Zn-36 and Zn-6 aptamer beacons and their elution capability in the presence of Zn2+. FIGS. 9A-9B depict a generic scheme of Zn²⁺ induced release of a molecular beacon from the capture oligonucleotide (FIG. 9A) and release of Zn-36 by Zn²⁺ (FIG. 9B). Beacon variants for Zn-36 are Zn-36m1 (FIG. 9C) and Zn-36m2-m6 (FIG. 9D). Elution of Zn-36m1-m6 is depicted in FIG. 9E. Beacon variants for Zn-6 are Zn-6m1 which has two possible stem loops (FIG. 9F), Zn-6m2 (FIG. 9G) and Zn-36m3-m6 (FIG. 9H). Elution of Zn-6m1-m6 in the presence of varying amounts of Zn2+ are depicted in FIGS. 9I-9J.

FIGS. 10A-10B depict the fluorescence responsivities of selected beacons. FIG. 10A shows fluorescence quenching in the presence of the capture oligonucleotide. The capture oligonucleotide q13 was present in a 2:1 (100 nM:50 nM) molar excess to beacons 14a or 16c. FIG. 10B shows a target-dependent increase in fluorescence. Complexes with the capture oligonucleotide were formed as in FIG. 10A, the target oligonucleotide OT2 was added in 2-fold excess, and the time-dependent development of signal was monitored. FIG. 10C shows the concentration-dependent response of beacon 14a to target oligonucleotide OT2. I is the signal-to-background ratio. These data are also shown as a function of concentration, rather than time, for the t=15 min time-point. FIG. 10D shows the concentration-dependent response of beacon 16c to target oligonucleotide OT2. These data are also shown as a function of concnetration for the t=15 min time-point.

FIGS. 11A-11C depict wavelength-shifting beacons. FIG. 11A shows the sequence and predicted structure of a wavelength-shifting beacon based on beacon 14a. Representations are as in FIG. 3C. The previously introduced fluorescein-dT is now labeled ‘F1’, while a second fluorphore (Texas Red) is ‘F2.’ There are two DABCYL moieties, at positions ‘Q1’ (previously introduced) and ‘Q2.’ FIG. 11B shows the fluorescence response at different wavelengths of beacon 14a to target OT2. The normalized signal-to-background ratio (percent change in fluorescence) is shown. FIG. 11C shows the fluorescence response at different wavelengths of beacon 16c to target OT2.

FIGS. 12A-12C depicts the change in fluorescence of the aptamer:quencher oligonucleotide complexes Zn-6m2 and Zn-36 m1 in solution upon the addition of Zn2+. FIG. 12A depicts a generic scheme showing different fluorescent states I-IV of the fluorescent aptamer beacon (I) in the presence of the quencher (II) and/or upon addition of Zn²⁺ (III-IV). FIG. 12B shows fluorescence of Zn-6m2 and FIG. 12C shows fluorescence of Zn-36m1 at each state I-IV.

FIGS. 13A-13B demonstrate concentration-dependent increase in fluorescence of Zn-6m2 apatmer beacon (FIG. 13A) and concentration-dependent deacrease in fluorescence of the Zn-6m2 fluorescent aptamer alone (FIG. 13B).

FIG. 14 depicts the specificity of Zn-6m2 for other metal ions.

### DETAILED DESCRIPTION OF THE INVENTION

In one embodiment of the present invention there is provided a method of selecting aptamer beacons in vitro comprising (a) generating a pool of single-stranded nucleic acid species which comprises a fluorphore F1 and a random insert of N nucleotides; (b) annealing the F1-labeled single-stranded nucleic acid species with a capture oligonucleotide which comprises an F1 quenching moiety Q1 to form a capture pool; (d) immobilizing the capture pool on a column; (e) eluting the capture pool with at least one target; (f) amplifying the F1-labeled single-stranded nucleic acid species comprising the eluate; and (g) repeating steps (a) through (f), wherein said selected F1-labeled single-stranded nucleic acid species comprise aptamer beacons.

In this embodiment the 5′-and 3′-regions of the single-stranded nucleic acid species may be constant regions. The F1 fluorphore may be attached within the 5′constant region. Also in this embodiment the capture oligonucleotide has a 3′- sequence complementary to a 5′-region in the single-stranded nucleic acid species. The capture oligonucleotide further may comprise biotin at the 3-end.

Further to this embodiment the method may comprise cloning the selected F1-labeled single-stranded nucleic acid species. Also further to this embodiment, the method may comprise the step of increasing a molar ratio of pool F1-labeled single-stranded nucleic acid species to target(s) as steps (a) through (f) are repeated. In another further embodiment the method may comprise eluting the capture pool with an eluent suitable to remove immobilized nucleic acid species binding to non-targets prior to step (e) and discarding the eluate. In an aspect of this further embodiment the non-target is an oligonucleotide and the eluent comprises a mixture of non-targets oligonucleotides which have no sequence similarity with the target oligonucleotide. In another aspect the non-target is a metal ion and the eluent comprises a buffer which excludes the metal ion.

Additionally in this embodiment the clones may be sequenced where clones having a motif of common residues at or near the 5′ end of the random insert comprise a family of aptamer beacons. In aspects of this embodiment the motif may have the sequence of SEQ ID NO: 35. The family of molecular beacons may comprise at least one of SEQ ID NOs.: 38-56. In another aspect the motif may have the sequence of SEQ ID NO: 129. The family of molecular beacons may comprise at least one of SEQ ID NOs.: 83-116.

In all aspects of this embodiment the random insert N in the nucleic acid species is about 100 nucleotides or less. In one particular aspect N is 20 nucleotides. The nucleic acid species may have the sequence shown in SEQ ID NO: 1. In another particular aspect N is 50 nucleotides. The nucleic acid species may have the sequence shown in SEQ ID NO: 64.

In all aspects the capture oligonucleotide may have the sequence of SEQ ID NO: 6 or SEQ ID NO: 68. In these aspects the target may be target an oligonucleotide, a metal ion, a peptide, a protein or a complex comprising a combination thereof. An example of a target oligonucleotide has the sequence of SEQ ID NO: 10. Examples of a metal are Zn²⁺, Mn²⁺, Mg²⁺, Co²⁺, or Ni²⁺. In all aspects the nucleic species may be DNA, RNA or modified DNA or modified RNA. Also in all aspects the aptamer beacons may have at least one of the sequences of SEQ ID NOs.: 36-59 or SEQ ID NOS: 83-116.

In a related aspect to this embodiment the 5′-end of the random insert of the F1-labeled single-stranded nucleic acid species may further comprise a fluorphore F2 which is different from fluorphore F1 and an F2 quenching moiety Q2 on the 5′ end of the 5′ single-stranded nucleic acid species. An example of F1 is fluorescein. An example of F2 is Texas Red, rhodamine red or tamra. The fluorescence quenchers Q1 and Q2 individually may be the fluorescence quenching moiety DABCYL or BHQ.

In another related embodiment there is provided an aptamer beacon selected by the method described supra. In this embodiment the characteristics of the aptamer beacons, the fluorphores F1 and F2, the fluorescent quenchers Q1 and Q2, and the specific aptamer beacons are as described. The aptamer beacons may comprise components having the sequences described supra.

In yet another embodiment of the present invention there is provided a method of detecting a ligand in solution comprising the steps of a) determining an initial level of fluorescence of a fluorphore F1 attached within the 5′ region of an aptamer beacon described supra; b) annealing the aptamer beacon with a capture oligonucleotide to form a captured beacon construct comprising an F1 quenching moiety where the quenching moiety Q1 quenches F1 upon binding; c) immobilizing the captured beacon construct; d) contacting the captured beacon construct with the solution; e) interacting the ligand with the captured beacon whereby the captured beacon is released from the capture oligonucleotide; and f) determining an increase in fluorescence of F1 from the quenched state of F1 upon the release of the captured beacon thereby detecting the ligand.

In this embodiment the 5′-region of aptamer beacon may be a constant region. The F1 fluorphore may be attached within the 5′ constant region. Also in this embodiment the capture oligonucleotide has a 3′-region sequence complementary to the 5′-region in the aptamer beacon. The capture oligonucleotide further may comprise biotin at the 3-end.

Further to this embodiment, the method comprises attaching a fluorphore F2 within a 5′ region of the random insert in the aptamer beacon, where the fluorphore F2 is different from fluorphore F1 and each of F1 and F2 exhibit a distinct color upon fluorescing; attaching an F2 quenching moiety Q2 on the 5′ end of the 5′ region of the aptamer beacon; detecting the fluorescent color of F2 prior to step d; quenching F2 with Q2 upon interacting the ligand with the captured beacon in step f; and detecting a change in fluorescent color from F2 to F1 upon the release of the captured beacon. An example of a fluorphore F2 is Texas Red, rhodamine red or tamra. An example of a fluorescence quenching moiety Q2 is DABCYL or BHQ. In all aspects of this embodiment the aptamer beacons, capture oligonucleotides, fluorphore F1 and quenching moiety Q1 are as described supra. The ligands may be those targets described supra.

In still another embodiment of the present invention there is provided a method of selecting a family of molecular beacons in vitro comprising the steps of (a) generating a pool of ssDNA having a random insert of N nucleotides between the 5′ and 3′constant regions where the ssDNA is labeled with a fluorphore F1 in the 5′ constant region; (b) annealing the F1-labeled ssDNA with a capture oligonucleotide complementary to the 5′ constant region of the F1-labeled ssDNA to form a capture pool where the capture oligonucleotide comprises a biotinylated 3′end and a 5′ end labeled with a fluorescence quenching moiety Q1 such that the quenching moiety Q1 is proximate to F1 thereby quenching F1; (d) immobilizing the capture pool on a column; (e) eluting the capture pool with at least one target ligand to release the F1-labeled ssDNA from the capture oligonucleotide whereby the F1-labeled ssDNA demonstrates an increase in fluorescence upon release from the capture oligonucleotide; (f) collecting an eluate comprising an F1-labeled ssDNA pool bound to the target ligand(s); (g) amplifying the F1-labeled ssDNA comprising the eluate; h) repeating steps (a) through (g) to select for the F1-labeled ssDNA pool bound to the target ligand; (i) cloning the selected F1-labeled ssDNA, and ( ) sequencing the clones, wherein clones having a motif comprising common residues at or near the 5′ end of the random insert comprise a family of molecular beacons.

In this embodiment the method may comprise the additional steps of increasing the ssDNA pool to target ligand molar ratio and of eluting the capture pool with an eluent suitable to remove immobilized nucleic acid species binding to non-targets as described supra. Additionally, the single or double fluorphore-labeled ssDNA, the fluorphores, the fluorescence quenching moieties, the capture oligonucleotides and the target ligands may be as described supra.

Furthermore, in all aspects of this embodiment the random insert N may be as described above. The motifs may have the sequence shown in SEQ ID NO: 35 or in SEQ ID NO: 129. The family of aptamer beacons selected by the method described supra may comprise at least one of the sequences shown in SEQ ID NOS.: 38-56 or in SEQ ID NOS. 83-116.

Additionally, in a related embodiment there is provided a aptamer beacon family selected by the method described supra. The aptamer beacons comprising the family including the characteristics of the aptamer beacons, the fluorphores F1 and F2, the fluorescent quenchers Q1 and Q2, the motif sequences and the specific sequences of the aptamer beacons are as described in the method of selecting such.

The following terms shall be interpreted according to the definitions set forth below. Terms not defined infra shall be interpreted according to the ordinary and standard usage in the art.

As used herein, the term “molecular beacon” shall refer to a hairpin stem structure with a fluor and a quencher at the two ends of the stem such that on binding a complementary oligonucleotide target, the structure opens up and a fluorescent signal is produced due to separation and consequent unquenching of the fluorphoree.

As used herein, the term “aptamer beacon” shall refer to a selected nucleic acid binding species (aptamer) which on target binding undergoes a conformational change that releases it from hybridization with a complementary capture oligonucleotide and allows it to exhibit a concomitant fluorescence increase due to separation of a fluorphore on the aptamer from a quencher on the capture oligonucleotide. Generally, generally, an aptamer beacon may be any oligonucleotide that upon binding of an analyte undergoes a conformational change that results in an optical or other signal such as electrochemical.

As used herein, the term “capture oligonucleotide” shall refer to an oligonucleotide which has sequence complementarity with the 5′ constant end of the DNA pool, a 5′ fluorescence quencher poised right opposite the internal fluorphore in the DNA pool and a 3′biotin such that it can be used to anneal, quench, and capture the pool on streptavidin column for affinity chromatography.

As used herein, the term “ligand” or “analyte” shall refer to any molecule that separates an oligonucleotide from its complement.

As used herein, the term “PCR” shall refer to the polymerase chain reaction that is the subject of U.S. Pat. Nos. 4,683,195 and 4,683,202 to Mullis, as well as other improvements now known in the art.

As used herein, the term “bases” shall refer to both the deoxyribonucleic and ribonucleic acids. The following abbreviations are used, “A” refers to adenine as well as to its deoxyribose derivative, “T” refers to thymine “U” refers to uridine, “G” refers to guanine as well as its deoxyribose derivative, “C” refers to cytosine as well as its deoxyribose derivative. A person having ordinary skill in this art would readily recognize that these bases may be modified or derivatized to optimize the methods of the present invention.

The following abbreviations are used herein. PCR: polymerase chain reaction; DABCYL: refers to the fluorescence quenching moiety 4-(4-dimethylaminophenylazo)benzoyl-linked to the 5′ end of a capture oligonucleotide or of a molecular beacon; BHQ: black hole quencher.

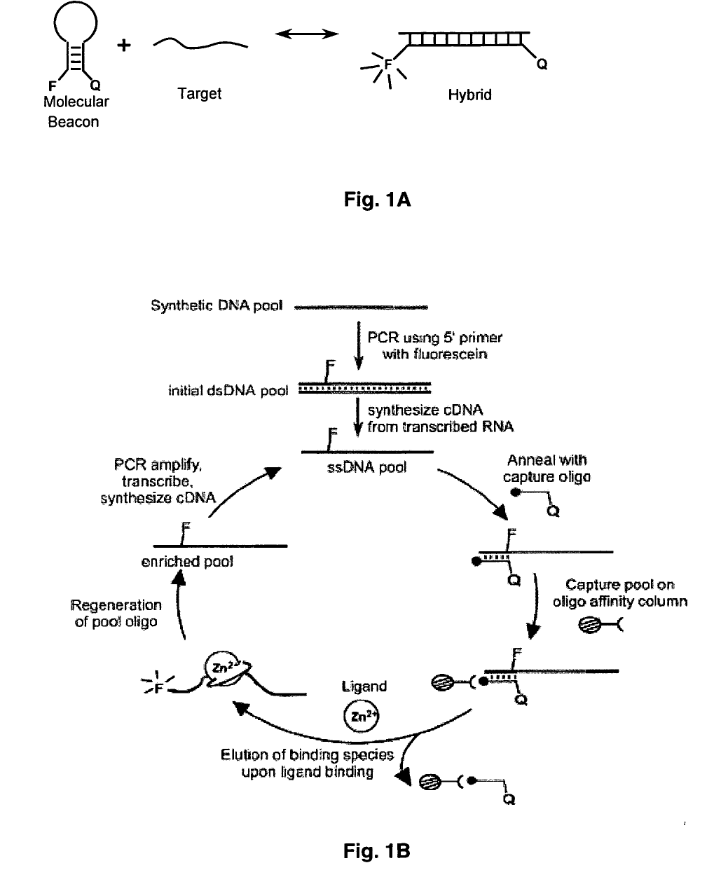

Provided herein is an in vitro combinatorial method for the direct selection of aptamer beacons that are responsive to oligonucleotide or other effectors, such as, but not limited to, Zn²⁺. The selection method is generalizable to any analyte or ligand that can be introduced into column chromatography. Most molecular beacons rely upon the hybridization of an oligonucleotide to the hairpin loop of a stem-loop structure, which in turn results in a conformational change that separates a fluorescent reporter from an adjacent quencher (FIG. 1A). The aptamer beacons comprise nucleic acids, for example, but not limited to, DNA are selected herein from a random sequence population. The selection method is based on analyte- or ligand-binding mediated conformational change and concomitant release of a fluorescently labeled DNA library from a complementary quencher oligonucleotide and thus couples generation of a fluorescent signal to binding.

Generally, the present invention provides a method whereby a pool of single stranded nucleic acid species is annealed to an oligonucleotide affinity column via one of its constant sequence regions (FIG. 1B). The hybridization poises a fluorescent reporter on the pool across from a quencher on the capture oligonucleotide. On addition of a target, such as an oligonucleotide or other analyte, e.g., a metal ion, any species which undergoes an target-dependent conformational change and in the process are released from the oligonucleotide affinity column, are collected, amplified, and carried into additional rounds of selection. The conformational change also concomitantly results in the fluor and quencher being separated from one another, and therefore leads to a target-dependent increase in fluorescence. Although the selected aptamer beacons may have properties similar to designed molecular beacons, the signaling mechanism requires that the aptamer beacon be separated from the capture oligonucleotide in the presence of a ligand or analyte. The method provides for application to a variety of ligand classes and contemplates new applications for the aptamer beacons selected by the method disclosed.

Design and optimization of aptamer beacon selection depends on the strength of the interaction between the oligonucleotide affinity column and the constant region of the pool. This determines whether and what kind of analyte- or ligand-dependent conformational change can be selected. The complementary interaction is such that the nucleic acid, e.g., DNA, pool is readily released from the affinity column following interactions with an analyte or ligand. Oligonucleotides of different lengths can capture and immobilize aptamer beacons, however insufficient length will not hold the DNA pool on the column and excessive length will not release DNA pools upon interaction with a target oligonucleotide or other analyte or ligand. Preferably, the capture oligonucleotide forms about 12 base pairs with an N or random region of up to 100 nucleic acids in the DNA pool. It is contemplated that the random region may be up to N100 or even larger provided the pool is synthetically accessible.

Selection of molecular beacons is skewed toward target- or ligand-dependent elution. A single-stranded DNA pool containing, N randomized positions, is used as a starting point for selection. A relatively small random sequence region makes any subsequent analysis of any selected molecular beacon mechanisms more straightforward. A fluorescent reporter F1 is introduced into the pool via a 5′ primer that contains a fluorescent thymidine residue at position 11 or 12 (T11, T12), e.g., fluorescein is conjugated to the 5 position of the nucleobase. Other fluorphores such as, but not limited to, Cascade blue, Alexa fluor 488 or Oregon green may substitute for fluorescein.

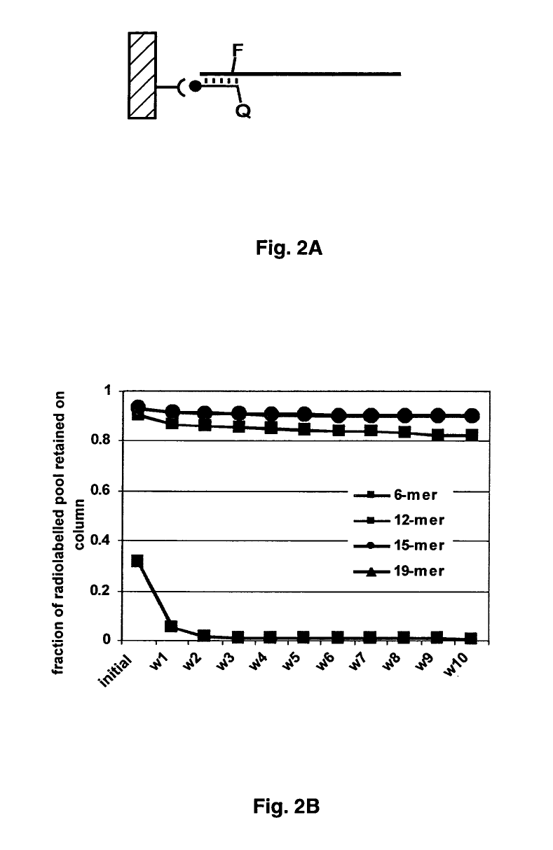

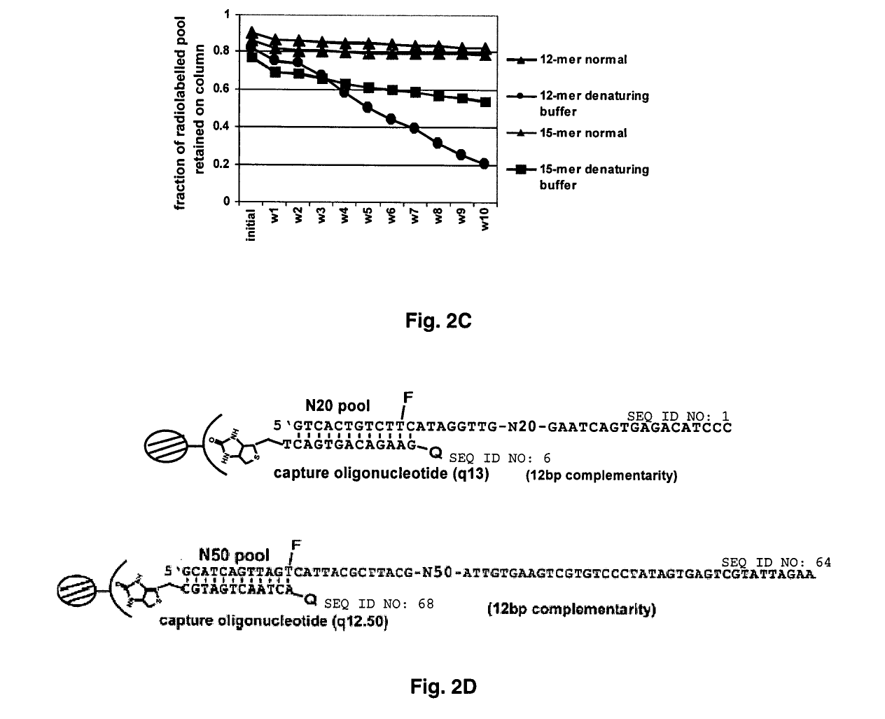

In each round of selection, the fluorescent, single-stranded DNA pool is annealed to the 12-residue capture oligonucleotide with subsequent immobilization of the duplex on a streptavidin-agarose column. The capture oligonucleotide also has a fluorescence quencher Q1, such as DABCYL, at its 5′ end. Other dark quenchers such as black hole quencher (BHQ) may be used. Upon hybridization, the DABCYL moiety is proximate to the fluorescein on the pool as shown in FIG. 2D. Hybridization can result in up to about 30-fold quenching of the fluorescein on the beacon construct.

Selection targets such as, but not limited to, 16-mer oligonucleotide targets or metal ions, e.g. Zn²⁺, are used to elute the immobilized pool from the column. Any eluted products have undergone a conformational change to release from the capture oligonucleotide. The species eluted by the target oligonucleotides are collected, amplified, and carried into the next round of selection. For elution with oligonucleotides, using two different oligonucleotide targets during selection may allow the evolution of either specificity or a lack of specificity for oligonucleotide targets and may determine whether some oligonucleotide targets were more effective in selecting beacons than others.

A negative selection step may be introduced into the selection process. For oligonucleotide targets a pre-elution step of incubating the pool with a set of about five 18-20-mer non-target oligonucleotides that do not resemble the two target oligonucleotides used for eluting the molecular beacons improves the eventual sequence selectivity of any selected beacons. For other analyte targets, such as a metal ion, the immobilized pool is incubated in selection buffer lacking the target metal ion. Selection is made with the specific targets. As selection progresses the ratio of the single-stranded nucleic acid pool to target is increased progressively to increase the stringency of the selection, i.e., the competition between aptamer beacons for targets is greater.

The mechanism of ligand or analyte-dependent elution uses a selected motif and a single residue from the constant region that could potentially form a stem-loop structure with the constant region. The stem-loop should in turn disrupt hybridization to the capture oligonucleotide. The aptamer beacons must have a sequence motif internally that is complementary to the 5′ constant end. The motif must be adjacent to the region complementary to the target. Incorporating these two features into a selected aptamer beacon provides for generality to a target ligand or analyte.

The eluted species function like molecular beacons demonstrating increases in fluorescence upon the addition of target oligonucleotides. The responsivities and kinetics of the selected aptamer beacons are comparable to many designed molecular beacons found in the prior art (1). The fluorescence responses of the selected molecular beacons were observable at room temperature and reached a stable level within 10-15 minutes.

Selected aptamer beacons may be synthesized enzymatically or chemically. However, chemically synthesized beacons show a smaller, i.e., 4- to 5-fold, as opposed to 10- to 20-fold, target-dependent increase in fluorescence. It is known that longer synthetic DNAs accumulate additional chemical lesions in contrast to the shorter primers for cDNA synthesis. As the length of synthetic DNA goes up, chemical lesions accumulate and its overall integrity and quality decreases (41). With chemical synthesis, extension with reverse transcriptase demonstrated that only 35% of the chemically-synthesized DNA did not contain lesions, as opposed to over 90% of the enzymatically-synthesized DNA (data not shown). Thus, enzymatic rather than chemical preparation of molecular beacons may provide a more robust performance.

Two separate conformational changes occur during the target-dependent activation of the selected aptamer beacons. The capture oligonucleotide is lost from the selected beacon construct and the selected beacon construct forms a hairpin stem. Therefore, a second quencher Q2, which may be identical to Q1, e.g., DABCYL or BHQ, may be introduced at the 5′ end of beacon constructs during chemical synthesis and a second fluorescent reporter F2, different from F1, e.g., Texas Red, rhodamine red or Tamra, may be appended at the 3′end of the fold-back motif which corresponds to the 5′end of the random insert. For example, F2 may be appended to a cytidine residue in the octamer motif at position 27 via post-synthetic chemical coupling.

In the absence of target, fluorescein would be quenched by the adjacent DABCYL on the capture oligonucleotide and Texas Red would fluoresce. In the presence of target, the conformational changes will bring the 5′ DABCYL on the beacon into apposition with Texas Red, and fluorescein would instead fluoresce. The doubly fluorescent selected color-switching beacons undergo a complete color change in which only one dye at a time is active in comparison to prior art (42) designed color-switching beacons that rely upon fluorescence resonance energy transfer between two dyes to yield a target-dependent change in color.

It is contemplated that the methods provided herein may provide for the selection of aptamer beacons. The ligands or analytes used for elution of species from the affinity column could be almost any molecule, from small ions to peptides to proteins to supramolecular complexes. Since any ligand-dependent release from column will of necessity also lead to a separation of a fluorescent reporter from a quencher, selection for binding and elution will select for signaling. Thus, at a minimum, the presence of ligands or analytes may be detected.

It is contemplated that the same selection strategy can be used to select aptamer beacons against other biologically relevant metal ions, including Mg²⁺, Ca²⁺, Fe²⁺, Cu²⁺, Mn²⁺, Co²⁺ and Ni²⁺. This is important, because while there are numerous other methods to generate fluorescent sensors against metal ions, both chemical and bio-sensors, these invariably are not generalizable and hence not amenable to high throughput generation of fluorescent sensors. Since the method presented herein is based on in vitro selection, it possibly offers the convenience of easy adaptability to different targets, with the added assurance that all selected binding species will of necessity have signaling capabilities. It may even be possible to select aptamer beacons that are responsive to a particular ionic state of a redox metal ion such as Fe²⁺ or Cu²⁺.

Additionally, the ability to select aptamer beacons may be useful when considering a complex target, such as a mRNA molecule. An aptamer beacon designed for mRNA of necessity will have regions designed into the beacon that are more or less accessible to regions in the mRNA, due to the formation of secondary and even tertiary structures and the binding of accessory proteins. However, it is contemplated that, using the method provided herein, molecular beacons may be selected de novo likely to interact with those portions of a mRNA that were most intrinsically accessible.

Furthermore, it is contemplated that the mechanism for signal transduction by aptamer beacons described herein also may yield applications. For example, to the extent that these structure-forming beacons require interactions with the free 3′end of a target oligonucleotide molecule, these beacons could be used to detect specifically the 3′ends of particular RNA molecules, including during RNA processing events. The more complex conformational changes that occur in selected molecular beacons, relative to designed molecular beacons, lend themselves more easily to more complex signaling modalities, such as the true two-color beacons constructed herein.

Furthermore, the conformational change undergone by the aptamer beacons in the presence of target is useful in its own right. For example, the conformational change itself could be coupled to other reporters, such as an electrochemical reporter. The electrochemical potential of the reporter changes as a result of a change in its chemical microenvironment due to conformational change of the aptamer beacon. In another application, the conformational change is useful as part of a microcantilever sensor, where mechanical stress is being measured.

Also, the property of release from immobilization present in the selected aptamer beacons may be employed in the design of oligonucleotide-specific actuators for nanoscale devices or nucleic acid-based machines (71-73). Yet another interesting possibility is the selection of metal ion responsive nucleic acid logic gates; for example, by carrying out selections with suitable combinations of metal ions, it might be possible to create logical operators such as “AND” gates. Nucleic acid based logic gates have thus far been made only by rational design.

The following examples are given for the purpose of illustrating various embodiments of the invention and are not meant to limit the present invention in any fashion.

### EXAMPLE 1

Synthetic DNA

All oligonucleotides were either made using an Expedite 8909 DNA synthesizer PE Biosystems (Foster City, Calif.) using synthesis reagents purchased from Glen Research (Sterling, Va.) or were ordered from Integrated DNA Technologies (Coralville, Iowa). Synthetic techniques use reported methodologies (32, 74).

N20 ssDNA Pool

A single-stranded DNA pool containing twenty randomized positions N20 (5′-GTCACTGTCTTCATAGGTTG-N20-GAATCAGTGAGACATCCC 3′) (SEQ ID NO: 1) was synthesized and was used as a starting point for in vitro selection. The pool was amplified using primers 20n.20 (5′-GTCACTGTCTTCATAGGTTG-3′) (SEQ ID NO: 2) and 38.20 (5′-TTCTAATACGACTCACTATAGGGATGTCTCACTGATTC-3′) (SEQ ID NO: 3), where the underlined residues indicate the non-transcribed portions of a T7 RNA polymerase promoter. A primer that contained biotin at its 5′ end, 18.20 (5′ Biotin-GGGATGTCTCACTGATTC 3′) (SEQ ID NO: 4), was used instead of 38.20 during later rounds of selection.

In experiments designed to optimize the length of the capture oligonucleotide, four different biotinylated oligonucleotides that were complementary to the N20 5′ constant region were utilized. These are 7.oa20 (5′ AGTGACT-Biotin 3′) (SEQ ID NO: 5), 13.oa20 (5′-GAAGACAGTGACT-Biotin-3′) (SEQ ID NO: 6), 15.oa20 (5′-TATGAAGACAGTGAC-Biotin-3′) (SEQ ID NO: 7) and 19.oa2O (5′-CAACCTATGAAGACAGTGA-Biotin-3′) (SEQ ID NO: 8). Oligonucleotides 7.oa20 and 13.oa20 had an additional residue at their 3′ends to reduce steric interference between biotin and streptavidin.

For selection experiments, a fluorescein-dT residue (Glen research, Sterling, Va.) was incorporated at the 11^{th} position of 20n.20 (20.11f :5′-GTCACTGTCTTCATAGGTTG-3′) (SEQ ID NO: 2), where the underlined residue corresponds to the site of insertion. DABCYL (Glen research, Sterling, Va.) was incorporated into the capture oligonucleotide q13.20 at its 5′ end (5′-DABCYL-GAAGACAGTGACT-Biotin-3′) (SEQ ID NO: 6). Two 16-mer oligonucleotides, OT1.20 (5′-ATGCGATCTAGTCTGC 3′) (SEQ ID NO: 9) and OT2.20 (5′-TAG CACGTCTGATCTC-3′) (SEQ ID NO: 10) were used as selection targets.

In those instances where negative selections were applied, five different oligonucleotides were used. These are RO1.18 (5′-GTA GTGCTCCGTGGATTG-3′) (SEQ ID NO: 11), R02.20 (5′-TCG AGGGAGAGCCATACCTG-3′) (SEQ ID NO: 12), RO3.20 (5′-TGCATGAG GATGCAGGATGC-3′) (SEQ ID NO: 13), RO4.20 (5′-ATTGATGAG TCTGACTGCCT-3′) (SEQ ID NO: 14), and RO5.19 (5′-GCGACTGGACAT CACGAGA-3′) (SEQ ID NO: 15).

Variants of selected beacons 14a and 16c were designed and synthesized to test the mechanism of oligonucleotide-dependent elution. For beacon 14a, these included oligonucleotides 14a.58 (5′-GTCACTGTCTTCATAGGTTTACTGTCAGAGATCAA CGTGCGAATCAGTGAGACATCCC-3′) (SEQ ID NO: 16), 14a.53a (5′-GTCACT GTCTTCTTGCGGTGACGAGATCAACGTGCGAATCAGTGAGACATCCC-3′) (SEQ ID NO: 17), 14a.53b (5′-TCACTGTCTTCATAGGTTGCGGAGATCAA CGTGCGAATCAGTGAGACATCCC-3′) (SEQ ID NO: 18), 14a.48 (5′-GTCACTGTCTTCATAGGGAGATCAACGTGCGAATCAGTGAGACATCCC-3′) (SEQ ID NO: 19), 14a.43 (5′-GTCACTGTCTTCGAGATCAACGTGCGAATCAG TGAGACATCCC-3′) (SEQ ID NO: 20) and 14a.42 (5′-GTCACTGTCTTCATAG GTTGCGGTGACGAGATCAACGTGCGA-3′) (SEQ ID NO: 21).

For beacon 16c these included oligonucleotides 16.58 (5′-GTCACTGTCTTCATAGGTTTACTGTCAGAGTCGGACGTGCGAATCAGTGA GACATCCC-3′) (SEQ ID NO: 22) and 16.48 (5′-GTCACTGTCTTCATAGGGAG TCGGACGTGCGAATCAGTGAGACATCCC-3′) (SEQ ID NO: 23). In addition, variant target oligonucleotides were assayed, including OT2b.20 (5′-TCGCACGTCTGATCTC-3′) (SEQ ID NO: 24), OT2c.20 (5′-TAGCACGTTGATCTC-3′) (SEQ ID NO: 25), OT2d.20 (5′-TATGTGATCTG ATCTC-3′) (SEQ ID NO: 26), OT2e.20 (5′-TAGCACGTCTGA-3′) (SEQ ID NO: 27), OT2g.20 (5′-ACGTCTGATCTC-3′) (SEQ ID NO: 28), OT2h.20 (5′-TAGCACGTCTGATCTCGTCA-3′) (SEQ ID NO: 29), and OT2j.20 (5′-TGATTAGCACGTCTGA-3′) (SEQ ID NO: 30). In order to test the generality of the signaling mechanism, a beacon cOT1 (5′-GTCACTGTCTTCATAGG TTGCGGTGACGCAGACTAATCGCGAATCAGTGAGACATCCC-3′) (SEQ ID NO: 31) was designed that was complementary to oligonucleotide target OT1. Additionally a beacon cOT3 (5′-GTCACTGTCTTCATAGGTTGCGGTG ACCACTCTTACATTGAATCAGTGAGACATCCC-3′) (SEQ ID NO: 32) was designed that was complementary to an unrelated 16-mer oligonucleotide target, OT3 (5′-TAATATGTAAGAGTG-3′) (SEQ ID NO: 33) or OT3b (5′-TCATATGCTAAGAGTG-3′) (SEQ ID NO: 34).

The wavelength-shifting molecular beacon constructs were 14mb.58 (5′-DABCYL-GTCACTGTCTTCATAGGTTGCGGTGACGAGATCAACGTGCGAAT CAGTGAGACATCCC-3′) (SEQ ID NO: 36) and 16mb.58 (5′-DABCYL-GTCACTGTCTTCATAGGTTGCGGTGACGAGTCGGACGTGCGAATCAGT GAGACATCCC-3′) (SEQ ID NO: 37). In addition to the 5′ DABCYL, these constructs contained a fluorescein-dT at position 11 and an amino modified-dC (Glen research, Sterling, Va.) at the 27^{th} position, as indicated by underlines.

N50 ssDNA Pool

To select aptamer beacons against Zn²⁺, in vitro selection experiments were initiated with a single-stranded DNA library containing 50 randomized positions (N50 pool). When compared to the previous selection for molecular beacons, a relatively larger pool was used to maximize the probability of selecting aptamers against zinc. A single-stranded DNA pool containing fifty randomized positions (N50; 5′-GCATCAGTTAGTCATTACGCTTACG-N50-ATTGTGAAGTCGTGTCCCTATA GTGAGTCGTATTAGAA-3′) (SEQ ID NO: 64) was synthesized using previously reported methodology,⁴² and was used as a starting point for in vitro selection. The pool was amplified using primers 25.50 (5′- GCATCAGTTAGTCATTACGCTTACG-3′) (SEQ ID NO: 65) and 38.50 (5′-TTCTAATACGACTCACTATA GGGACACGACTTCACAAT-3′) (SEQ ID NO: 66), where the underlined residues indicate the non-transcribed portions of a T7 RNA polymerase promoter. A primer that contained biotin at its 5′ end, 18.50 (5′ Biotin-GGGACACGACTTCACAAT-3′) (SEQ ID NO: 67), was used instead of 38.50 during later rounds of selection.

For selection experiments, a fluorescein-dT residue (Glen Research, Sterling, Va.) was incorporated at the 12^{th} position of 25.50 as 25a.50 (5′-GCATCAGTTAGTCATTACGCTTACG-3′) (SEQ ID NO: 65) so that the fluorphore will be in direct apposition to the quencher moiety on the capture oligonucleotide. The underlined residue corresponds to the site of insertion. DABCYL (Glen research, Sterling, Va.) was incorporated into the capture oligonucleotide q12.50 at its 5′ end (5′-DABCYL-ACTAACTGATGC-Biotin 3′) (SEQ ID NO: 68).

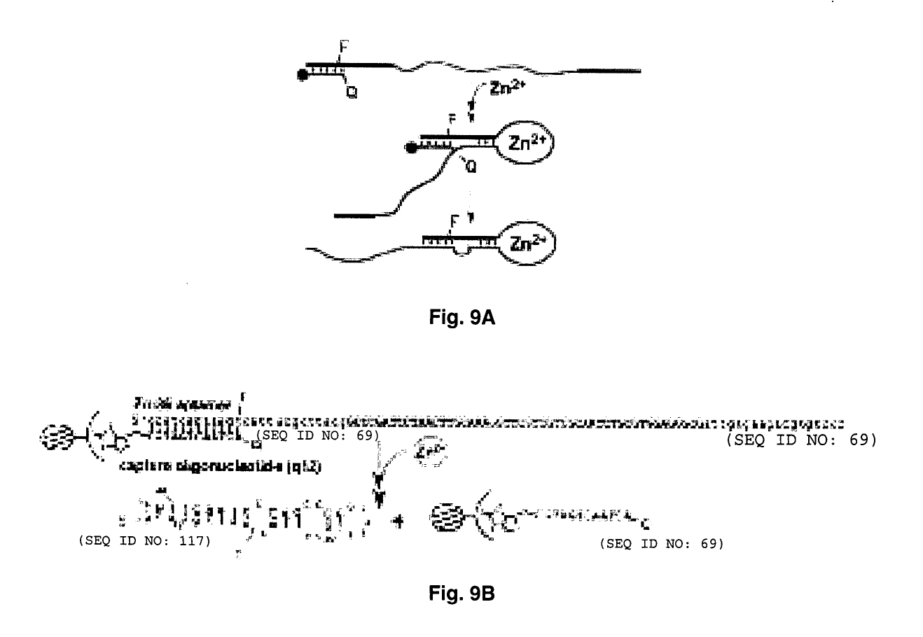

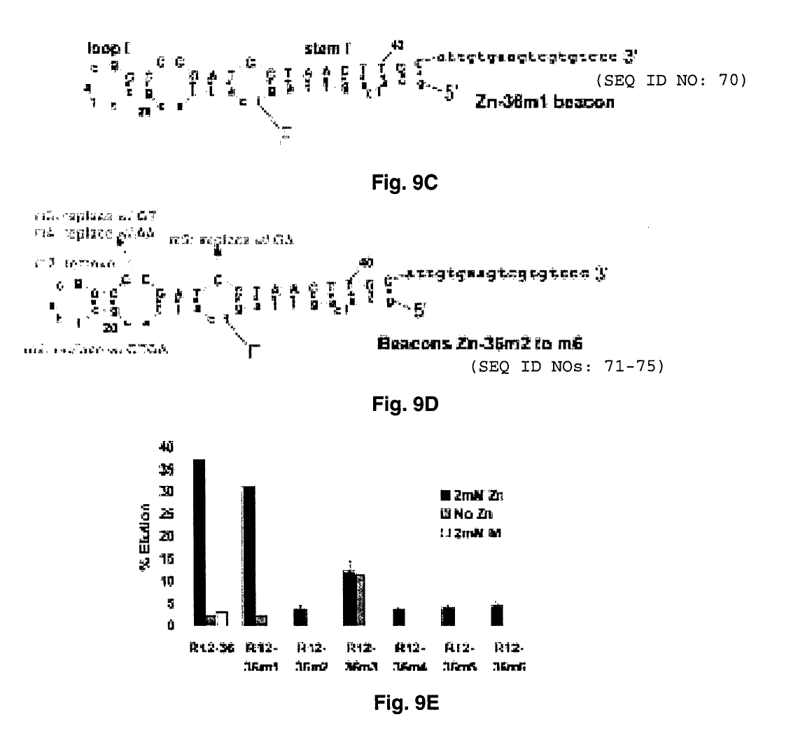

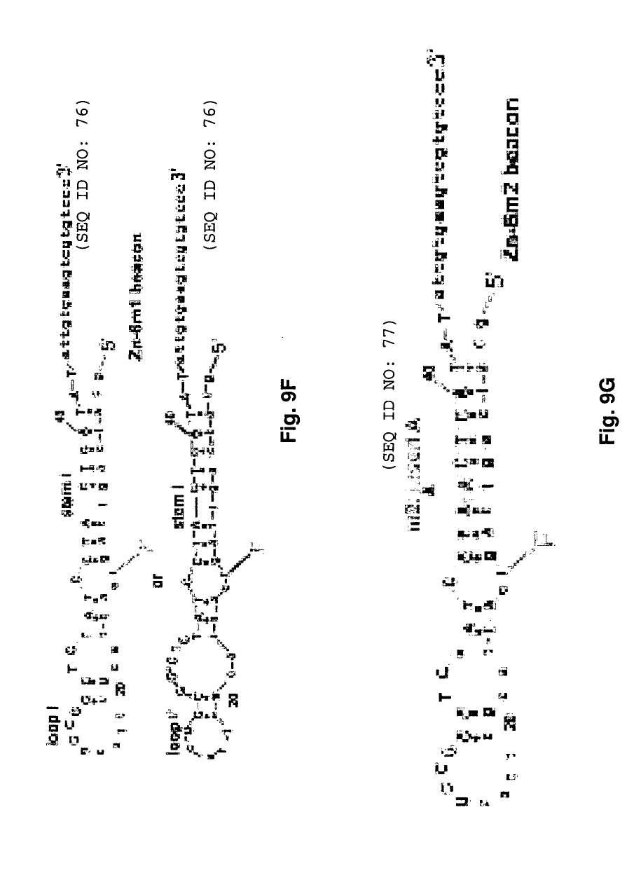

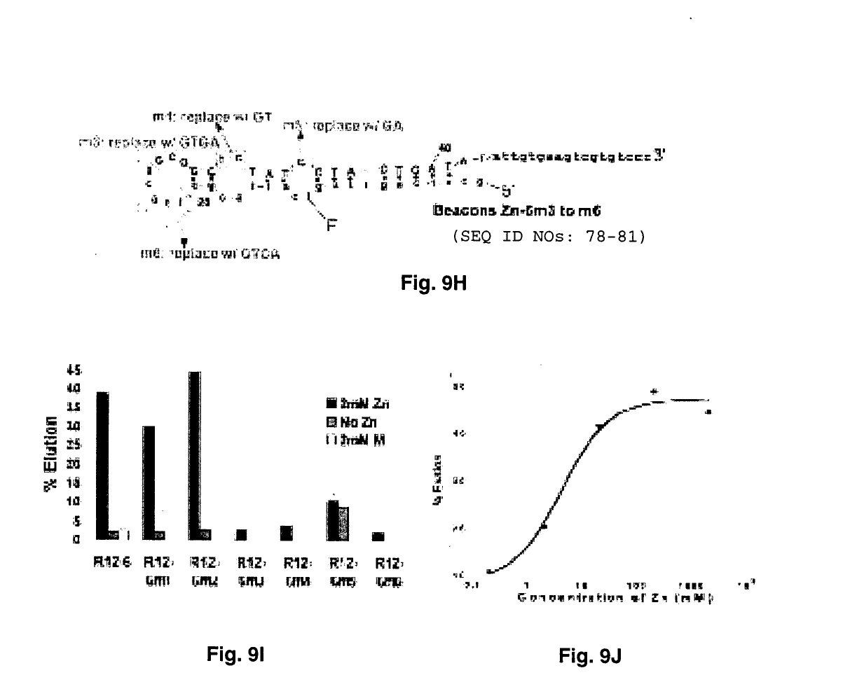

To test the mechanism of Zn²⁺-dependent elution, variants of selected beacons Zn-6 and Zn-36, were designed and synthesized. For beacon Zn-6, these included the oligonucleotides Zn-6m1 (SEQ ID NO: 69), Zn-6m2 (SEQ ID NO: 70), Zn-6m3 (SEQ ID NO: 71), Zn-6m4 (SEQ ID NO: 72), Zn-6m5 (SEQ ID NO: 73) and Zn-6m6 (SEQ ID NO: 74) and for beacon Zn-36, the oligonucleotides Zn-36m1 (SEQ ID NO: 75), Zn-36m2 (SEQ ID NO: 76), Zn-36m3 (SEQ ID NO: 77), Zn-36m4 (SEQ ID NO: 78), Zn-36m5 (SEQ ID NO: 79), Zn-36m6 (SEQ ID NO: 80) and Zn-36m7 (SEQ ID NO: 81). The sequences of these various oligonucleotides have been listed in FIGS. 9B-9D and 9F-9H.

A doped sequence pool, D22, designed based on the minimized aptamer beacon, Zn-6 m 2, had the sequence 5′-gcatcagttagtcattacgcttacgGCGGCTCTATCCTAACTGATATattgtgaagtcgtgtccc-3′ (SEQ ID NO: 82), where the residues in upper case indicate the positions which were doped at 55% wild type, and 15% of each non-wild type residue. The primers used to amplify this pool were the same as those for N50.

### EXAMPLE 2

DNA Pool Construction

Single-stranded N20 or N50 fluorescently labeled DNA pools were generated by a combination of chemical synthesis, PCR amplification, in vitro transcription, and reverse transcription.

N20 Pool

Following chemical synthesis, the N20 DNA pool was purified on an 8% denaturing polyacrylamide gel. The gel-purified pool, 32 micrograms, was amplified in a 25 ml PCR using the non-fluoresceinated primers 20n.20 and 38.20. Only 15% of the initial pool could be extended by Taq DNA polymerase. However, since the theoretical pool size was relatively small (4²⁰=1.1*10¹² molecules), there should have been an average 130 copies of each species in the pool. The PCR yielded over 2,000 pool equivalents.

Primer 38.20 contained a T7 RNA polymerase promoter and sixty-five pool equivalents were transcribed using the Ampliscribe T7 In vitro Transcription kit (Epicenter Technologies, Madison, Wis.). The resultant RNA was gel-purified on an 8% denaturing polyacrylamide gel. Greater than 2,000 RNA pool equivalents were reverse-transcribed with SuperScript II reverse transcriptase (Invitrogen, Carlsbad, Calif.) using the fluoresceinated 5′ primer, 20.11f, in a 500 μl RT reaction. The reverse transcription reaction was performed according to the protocol provided with the SuperScript II RT enzyme (Invitrogen, Carlsbad, Calif.) using 5 μg of RNA template per 20 μl RT reaction. The cDNA:RNA duplexes were digested with RNase A and Ribonuclease H (37° C., 25 min; 80° C., imin; 37° C., 30 min) to remove template RNA, and the remaining fluoresceinated cDNA was purified on an 8% denaturing polyacrylamide gel.

N50 Pool Construction

Following chemical synthesis, the N50 DNA pool was purified on a 6% denaturing polyacrylamide gel. The gel-purified pool (440 micrograms) was amplified by polymerase chain reaction using the non-fluoresceinated primers 25.50 and 38.50. The amplified DNA pool (5.5 * 10¹⁴ molecules) was then transcribed using the Ampliscribe T7 In Vitro Transcription kit (Epicenter Technologies, Madison, Wis.) and the resultant RNA, gel-purified on an 8% denaturing polyacrylamide gel. The RNA pool was subsequently reverse-transcribed with SuperScript II reverse transcriptase (Invitrogen, Carlsbad, Calif.) using the fluoresceinated 5′ primer, 25a.50 and the cDNA:RNA duplexes digested with RNase A and Ribonuclease H (37° C., 25 min; 80° C., imin; 37° C., 30 min) to remove template RNA. The remaining fluoresceinated cDNA was purified on an 8% denaturing polyacrylamide gel.

### EXAMPLE 3

Optimization of Capture Oligonucleotide Length

Oligonucleotide affinity columns of different lengths were constructed, and their abilities to capture and release DNA pools containing complementary constant regions were determined. The nascent, single-stranded DNA pool was 5′ end-labeled with T4 polynucleotide kinase (Invitrogen, Carlsbad, Calif.) and [γ-³²P]ATP (2.0 mCi, 7000 Ci/mmol, ICN Biomedicals, Costa Mesa, Calif.). Following gel purification, 50 pmoles of the labeled pool were annealed with 100 pmoles of the biotinylated capture oligonucleotide 7oa.20 in a 20 μl reaction volume. The annealing reaction was heated at 94° C. for 30 sec and 45° C. for 90 sec and was then cooled to room temperature. The annealing reaction was diluted to 500 μl using binding buffer (20 mM Tris, pH 7.5, 150 mM NaCl, 10 mM MgCl₂) and the bound pool was captured on streptavidin-agarose (Sigma-Aldrich, St. Louis, Mo.) over a period of 25 minutes (FIG. 2A).

The streptavidin-agarose was transferred to a column (Bio-Rad, Hercules, Calif.) and the amount of radioactivity in the eluant was determined using a scintillation counter. The column was washed 10 times with 1 mL of binding buffer, fractions were collected and the amount of radioactivity in the fractions was determined. Similar assays were carried out with the other capture oligonucleotides 13.oa20, 15.oa20 and 19.oa20 (FIG. 2B). The total amount of pool captured on each oligonucleotide was also determined by carrying out similar experiments in parallel, except that the columns were eluted with denaturing buffer (7M urea, 0.1M sodium citrate, 3 mM EDTA, pH 5) (FIG. 2C).

The capture oligonucleotides were complementary to 6-, 12-, 15-, or 19-residues in the constant region of the pool. Captured pools were eluted with 7M urea. The shortest hybridization interaction, 6-base-pairs, was insufficient to hold the pool on the column at room temperature. However, capture oligonucleotides that formed 12-, 15-, or 19-base-pairs with the pool were all effective in immobilization. The elution profiles of the 12- and 15- residue capture oligos were then generated. While a 12-base-pair interaction could be readily disrupted by a urea wash, the 15-base-pair interaction was too strong to immediately allow elution. The 12-base-pair capture oligonucleotide q13 including a fluorescence quencher (DABCYL) at its 5′ end is used for further selection of molecular beacons from the N20 pool of ssDNA. Similarly the 12-base-pair capture oligonucleotide q12.50 is used for selection of molecular beacons from the N50 pool of ssDNA (FIG. 2D).

### EXAMPLE 4

In vitro Selection from N20 and N50 DNA Pools

N20 Selection

To initiate the selection, the fluoresceinated, single-stranded N20 DNA pool, i.e., 100 pool equivalents, was annealed with a two-fold molar excess of the biotinylated capture oligonucleotide q13.20 in 100 μl water. The annealing reaction was heated to 94° C. for 30 sec and 45° C. for 90 sec and was then cooled to room temperature. The capture oligonucleotide and bound pool were immobilized on streptavidin-agarose (Sigma-Aldrich, St. Louis, Mo.) and transferred to a column. The column was equilibrated with selection buffer (20 mM Tris, pH 7.5, 150 mM NaCl, 10 mM MgCl₂) by repeated washing. Upon hybridization, the DABCYL was in proximity to the fluorescein on the pool as shown in FIG. 2D.

An equimolar mixture of the two oligonucleotide targets in selection buffer was heat-denatured, cooled to room temperature, added to the column containing the immobilized pool, and allowed to react for 10 minutes at room temperature with occasional mixing. The column was then drained and washed three times with 200 μl aliquots of binding buffer. All the eluates were collected and the eluted DNA was precipitated with ethanol. The eluted DNA was amplified by the PCR, transcribed to generate RNA, and reverse-transcribed. The cDNA was purified by RNase digestion and gel purification, as before, and was used for the next round of selection.

Seven rounds of selection and amplification were performed as described above, except that the number of washes that occurred prior to elution was successively increased. The pool to target ratio was also increased from 1:1 in the first three rounds to 2:1 in the next four rounds. The increased competition of pool molecules for targets should have increased the stringency of the selection.

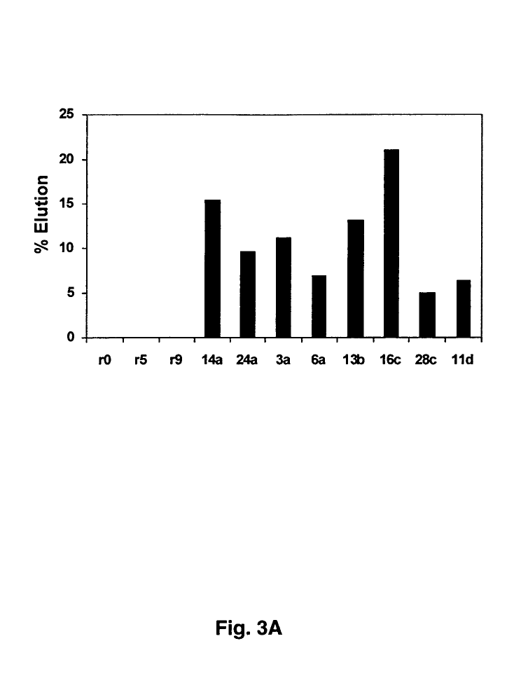

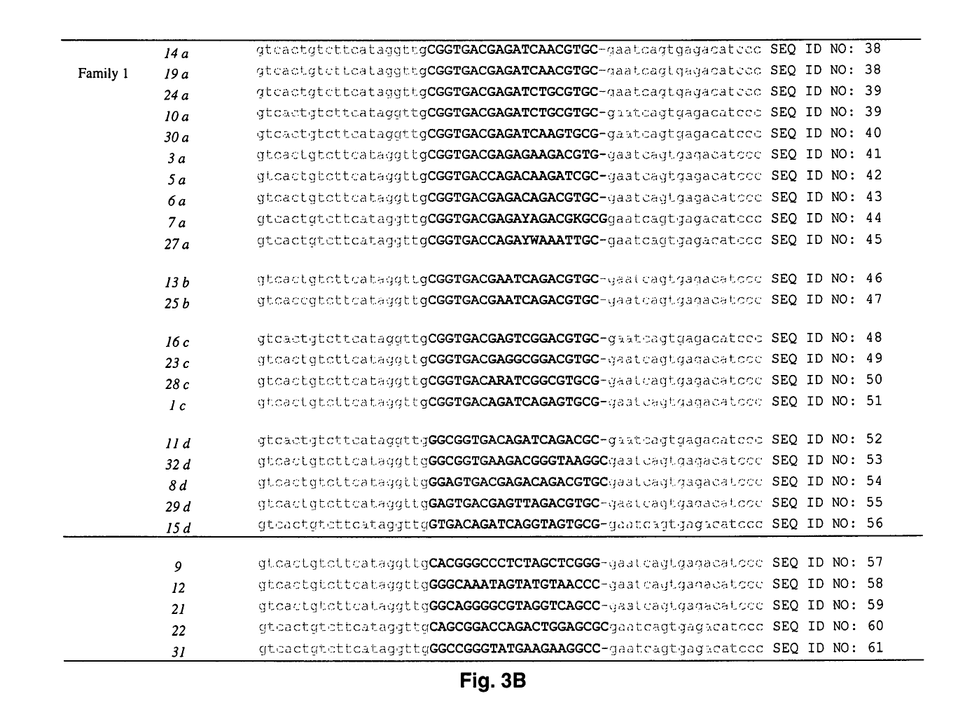

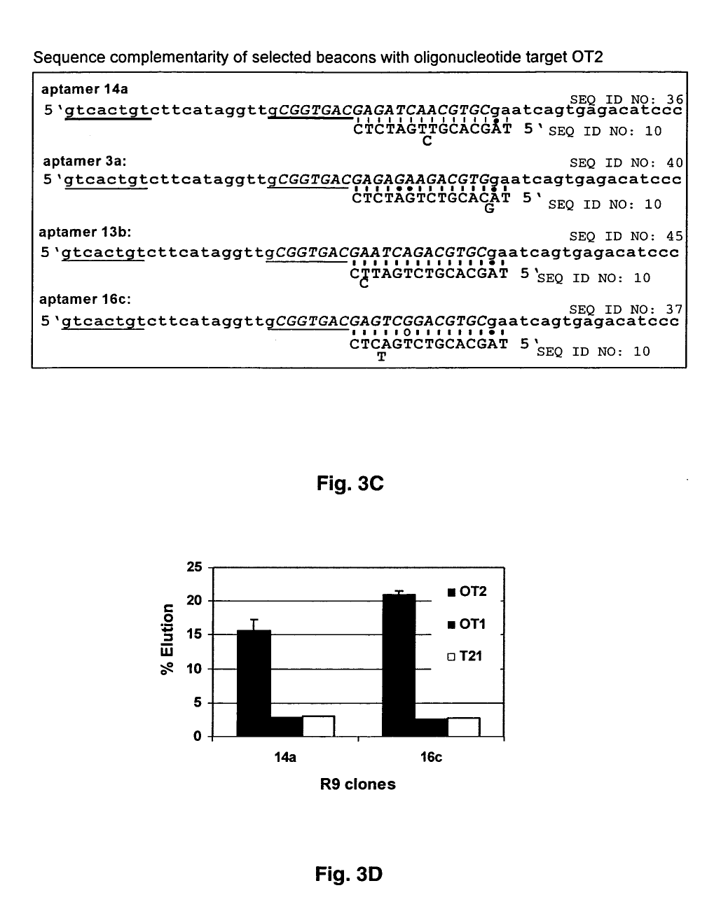

During the eighth and ninth rounds of selection, a negative selection step was introduced. The immobilized pool was first incubated with a mixture of 5 different oligonucleotides, i.e., RO1.18, RO2.20, RO3.20, RO4.20, and RO5.19, that bore no sequence similarity to the targets. Any pool members that eluted with these random oligonucleotides were discarded. The remaining pool was then eluted with the two target oligonucleotides, except that the pool to target ratio was again increased to 5:1 in the eighth round and to 10:1 in the ninth round (FIG. 3A).

Instead of amplifying selected molecules via transcription and reverse transcription, DNA species from Rounds 7 and 8 were PCR-amplified with the biotinylated primer 18.20 and the fluoresceinated primer 20.11f, the double-stranded PCR products were captured on streptavidin-agarose, and fluoresceinated, single-stranded DNA molecules were eluted with 0.2N NaOH. These eluates were immediately neutralized by adding 3M NaOAc at pH 5.2 and precipitated with ethanol. While this latter, faster method for single-strand DNA preparation could have been used in earlier rounds as well, the reverse transcription method was adopted because it gave consistently better yields of single stranded DNA, often >70% of input, and therefore helped to maximize the recovery of the amplified single strand DNA pool during the early rounds of selection. Ultimately, the Round 9 selected pool was cloned (TA Cloning kit, Invitrogen, Carlsbad, Calif.) and sequenced using the Dye Terminator Cycle Sequencing kit (Beckman Coulter, Fullerton, Calif.) and a CEQ 2000 XL DNA sequencer (Beckman Coulter; 30).

Twenty-six individual beacons from Round 9 were cloned and sequenced (FIG. 3B). Twenty-one of the selected beacons contained from five to seven common residues at or near the 5′ end of the random region or insert and are designated as Family 1. Some beacons differed from one another by only one residue and may have been derived from a common ancestor, e.g., compare clones 16c and 23c. Other beacons shared a core of sequence similarities, but otherwise differed at several positions, e.g., compare clones 19a, 24a and 3a. A number of outlier sequences still were present in the population. Selected sequences that contained the heptamer motif generally were complementary to OT2 but not to OT1 (FIG. 3C). The elution profiles of molecular beacons specifically eluted with OT2, OT1 or another unrelated oligonucleotide target, T21 are shown in FIG. 3D.

N50 Selection

The selection was initiated by annealing the fluoresceinated, single-stranded N50 DNA pool (1.5 pool equivalents) with a two-fold molar excess of the biotinylated capture oligonucleotide q12.50 having a DABCYL on the 5′ end in 50 ml 1× selection buffer (50 mM HEPES pH7.0, 300 mM NaCl, 0.5 mM MgCl₂). The annealing reaction was heated to 94° C. for 30 sec and 45° C. for 90 sec, and was then cooled to room temperature.

Hybridization of the pool with the capture oligonucleotide brought the fluorescein in close proximity to the DABCYL, and also allowed immobilization of the pool on streptavidin- agarose (FIG. 2D). The capture oligonucleotide and bound pool were immobilized on streptavidin-agarose (Sigma-Aldrich, St. Louis, Mo.) and transferred to a column. The immobilized pool was washed several times with selection buffer to remove pool members not bound or poorly bound on the column and then, incubated with the selection buffer containing 2 mM Zn²⁺ (50 mM HEPES pH7.0, 300 mM NaCl, 0.5 mM MgCl₂, 2 mM ZnCl₂) for 25 minutes at room temperature with occasional mixing. The column was then drained and washed three times with 400 ml aliquots of binding buffer. All the eluates were collected and the eluted DNA was precipitated with ethanol. The eluted DNA was amplified by the PCR, and strand separated via either transcription-reverse transcription-RNA degradation, or, by NaOH mediated separation of immobilized biotinylated double stranded DNA from streptavidin column. The amplified single stranded DNA was used for the next round of selection.

Since the only difference between the normal selection buffer and the buffer during binding-incubation is the presence of Zn²⁺, the eluted sequences would presumably have to be species which undergo a zinc dependent conformational change that releases them from the capture oligonucleotide. The selection buffer was also designed to contain a lower concentration of Mg²⁺ than usually used for selections, such as was used during selection from the N20 ssDNA pool, so that the dependence for Zn²⁺will be selective.

However, sequences which can undergo target independent conformational changes can also auto-elute from the oligonucleotide column and hence could exist in the population. From the fourth round of selection onwards, a negative selection step was introduced. The immobilized pool was incubated in selection buffer lacking zinc for ˜20 minutes prior to incubation in the presence of Zn²⁺, to remove species which have a propensity to auto-elute in a zinc-independent fashion. As such, the number of washes carried out prior to elution was successively increased.

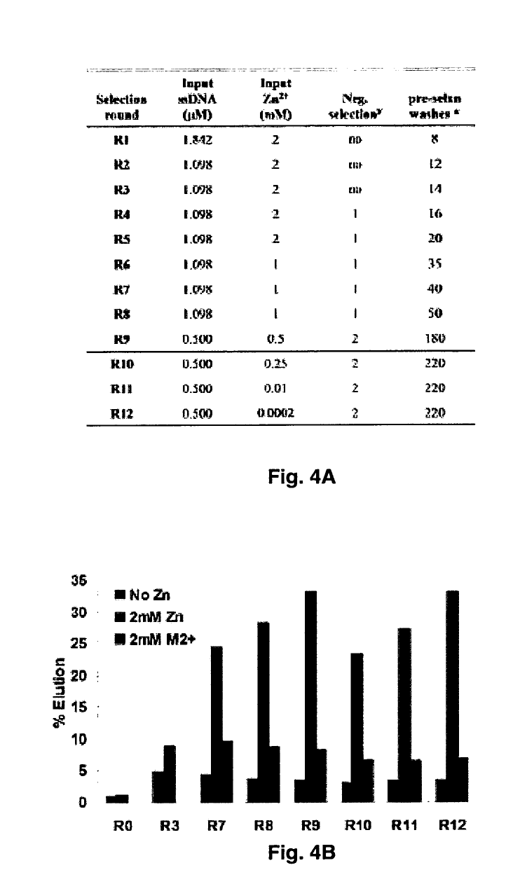

Nine rounds of selection and amplification were performed at relatively low stringency in an effort to select binding species (FIG. 4A). The binding affinities of aptamers for small molecule targets are often in the micromolar to millimolar range (75-75). Since the round 9 pool showed a significant improvement in target dependent elution, the stringency was increased thereafter to select high affinity species.

During the ninth round of selection, an additional negative selection step was introduced to improve the metal ion specificity of the selected aptamers. The immobilized pool was pre-incubated with a mixture of other transition metal ions excluding Zn²⁺ (50 mM HEPES pH7.0, 300 mM NaCl, 0.5 mM MgCl₂, 2 mM MnCl₂, 2 mM NiCl₂, 2 mM COCl₂). The zinc concentration was kept high during the first nine rounds of selection, but was progressively decreased to 2.5 fold below the pool concentration to increase competition between the pool molecules for the Zn²⁺ ions and thus, increase the stringency of the selection.

The target dependent elution did not improve much further during the next three rounds of selection (FIG. 4B). After round 12, the selected pool was cloned (TA Cloning kit, Invitrogen, Carlsbad, Calif.) and sequenced using the Dye Terminator Cycle Sequencing kit (Beckman Coulter, Fullerton, Calif.) and a CEQ 2000 XL DNA sequencer (Beckman Coulter) (77).

For the doped selection, the first round was initiated with 146 pmol of single stranded DNA pool (8.8×10¹³ molecules), and subsequent rounds were performed using 62.5 pmol of the DNA pool (3.7×10¹³ molecules). Overall, the procedure was similar to that used for the initial selection, except that from round 2 onwards, the incubation times were now reduced to 10 min instead of 25 min and the pool to target ration was progressively increased over six rounds from 2:1 to 20:1.

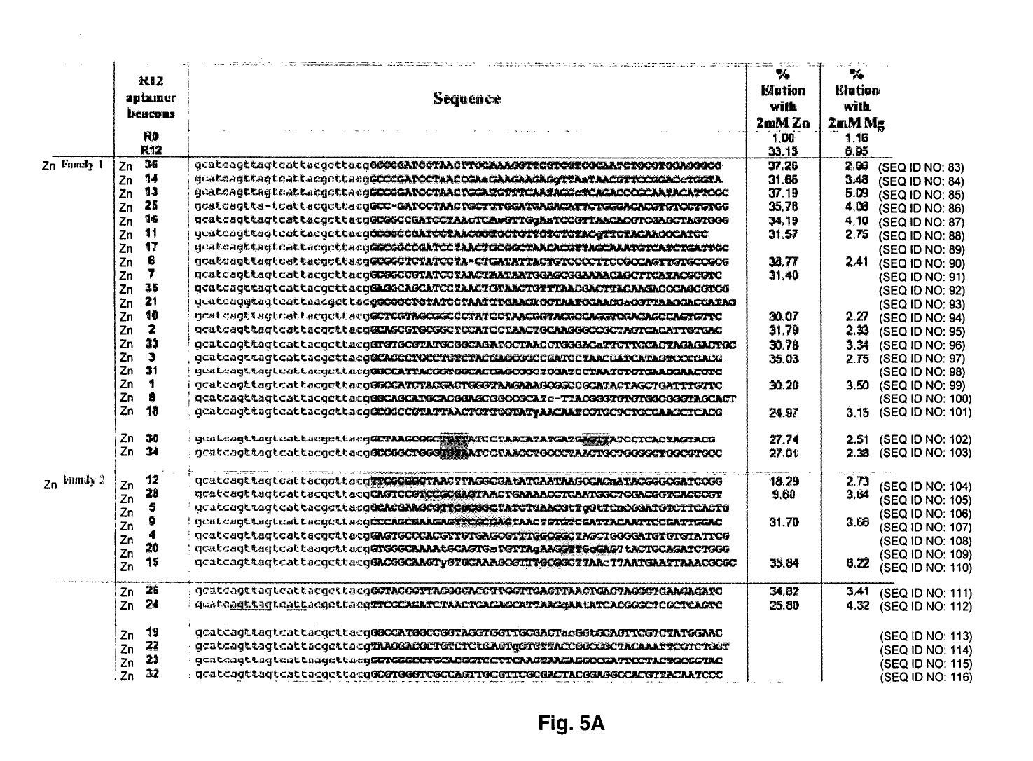

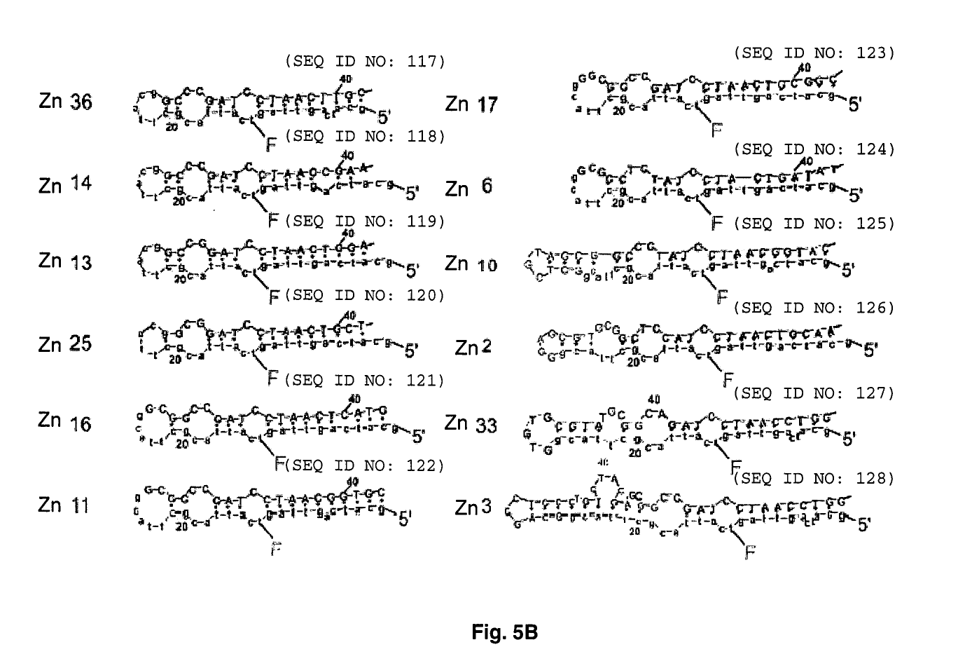

The round 12 pool was cloned, and thirty four individual aptamers were sequenced (FIG. 5). Twenty-one of the selected beacons were found to have eight to fourteen common residues (Family Zn1). Interestingly, apart from these fourteen residues, the remaining 36 bases in the random region were all different among the members of this family. A second, smaller family of beacons was also observed which contained 5-6 residues common with family Zn1 and other additional 5-8 common residues (Family Zn2). In addition, a few other sequences also had the same 5 residue motif as present in families Zn1 and Zn2, but with no other similarities. A number of outlier sequences were also still present in the population.

The program Mfold was used to model the secondary structure of the selected aptamer beacons (78). All the family 1 aptamer beacons could potentially fold to form a similar secondary structure (FIG. 5B). When the selected beacons were assayed for their dependence on Zn²⁺, a majority of them showed high Zn²⁺ dependent elution abilities, and were also specific for Zn²⁺ (FIG. 5A). The two beacons, Zn-6, and Zn-36, which showed the maximum elution with Zn²⁺, and which were most specific for Zn²⁺, were chosen for further analysis.

The pentamer motif, TAACT (SEQ ID NO: 129) which was found in 30 out of 34 of the selected beacons potentially can form a stem loop structure with the 5′constant region. In the family Zn1 aptamer beacons, the additional common residues of the 14-mer motif lead to a bulged stem-loop structure in which the sequence of the terminal helix and the two continuous central bulges is almost invariant (region highlighted in yellow in FIG. 5B). Since the formation of this secondary structure necessitates displacement of the capture oligonucleotide, this could be a possible mechanism for zinc dependent elution. The aptamer beacons are likely undergoing a conformational change on zinc binding which leads to alternate stem formation with the 5′ constant region and hence, release of the complementary capture oligonucleotide.

### EXAMPLE 5

Binding Assays for N20 and N50 ssDNA

Following specific Rounds, the amplified, single-stranded DNA pools were 5′ end-labeled using T4 polynucleotide kinase (Invitrogen, Carlsbad, Calif.) and [γ-³²P]ATP (2.0 mCi, 7000 Ci/mmol, ICN Biomedicals, Costa Mesa, Calif.). Binding assays were performed in a manner similar to the selection experiments themselves, except that fractions were collected for scintillation counting.

N20 ssDNA

The amplified, single-stranded DNA pools were 5′ end-labeled following Rounds 5, 7 and 9. 50 pmoles of gel-purified, labeled single-stranded DNA pool were annealed with 100 pmoles of the capture oligonucleotide q13 in a 50 μl reaction, as described above. The radiolabeled pool was immobilized on 60 μl of streptavidin-agarose (Sigma-Aldrich, St. Louis, Mo.) and the unbound fraction was collected. The column was washed three times with 300 μl of selection buffer (20 mM Tris, 7.5, 150 mM NaCl, 10 mM MgCl₂) and the washes were again collected.

A mixture of the two oligonucleotide targets in binding buffer was prepared such that each target would be at the same final concentration as the pool. The oligonucleotide targets were heat-denatured and added to the immobilized pool in a total volume of 200 μl. As with the selections, binding reactions were incubated for 10 minutes prior to washing the column two times with 10× column volumes of binding buffer. All the eluants and the remaining solid resins were preserved, radioactivity was quantitated using a scintillation counter and the proportions of the pools that were specifically eluted by target oligonucleotides were determined.

N50 ssDNA

The amplified, single-stranded DNA pools were 5′ end-labeled following Rounds 3, 7, 8, 9, 10, 11, and 12. 40 pmoles of gel-purified, labeled single-stranded DNA pool were annealed with 80 pmoles of the capture oligonucleotide q12.50 in a 50 ml reaction, as described above. The radiolabeled pool was immobilized on 50 ml of streptavidin-agarose (Sigma-Aldrich, St. Louis, Mo.) and the unbound fraction was collected. The column was washed three times with 300 ml of selection buffer (50 mM HEPES pH7.0, 300 mM NaCl, 0.5 mM MgCl₂) and the washes were again collected.

A 2 mM solution of Zn²⁺ in the binding buffer (50 mM HEPES pH7.0, 300 mM NaCl, 0.5 mM MgCl₂, 2 mM ZnCl₂) was added to the immobilized pool in a total volume of 200 ml. As with the selections, the binding reactions were incubated for 25 minutes prior to washing the column two times with 500 ml of binding buffer. All the eluants and the remaining solid resins were preserved, radioactivity was quantitated using a scintillation counter, and the proportions of the pools that were specifically eluted by target oligonucleotides were determined.

### EXAMPLE 6

Binding Assays with Beacon Variants for N20 ssDNA

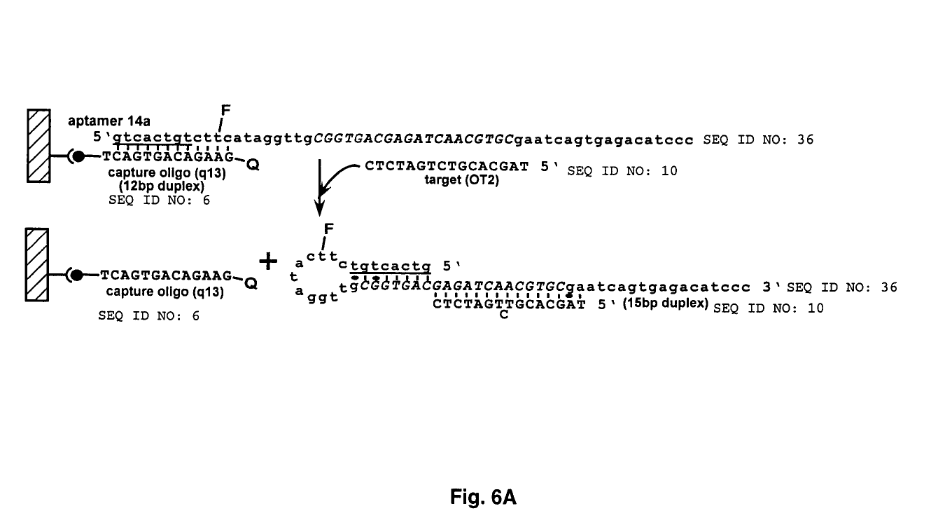

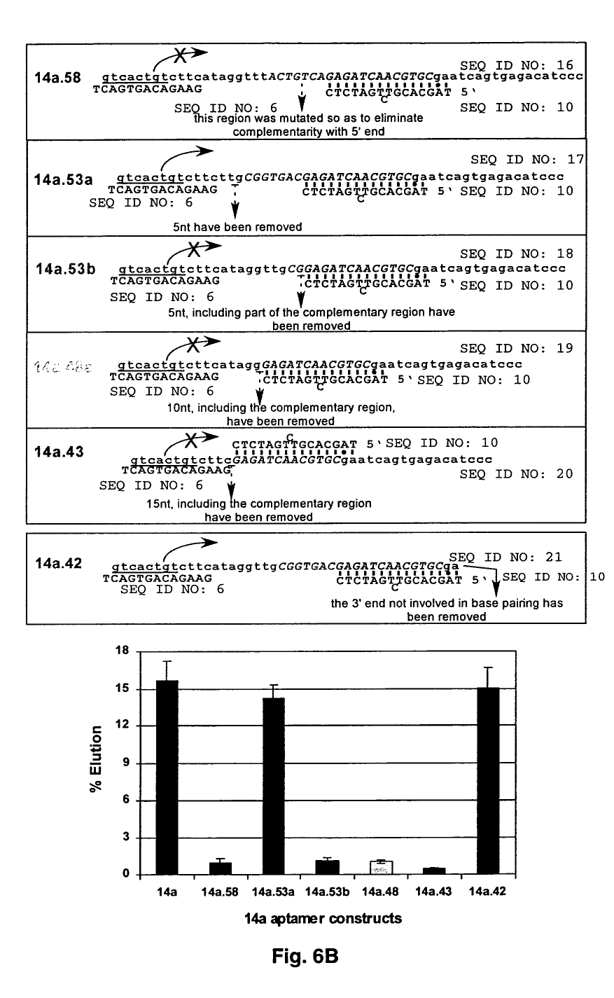

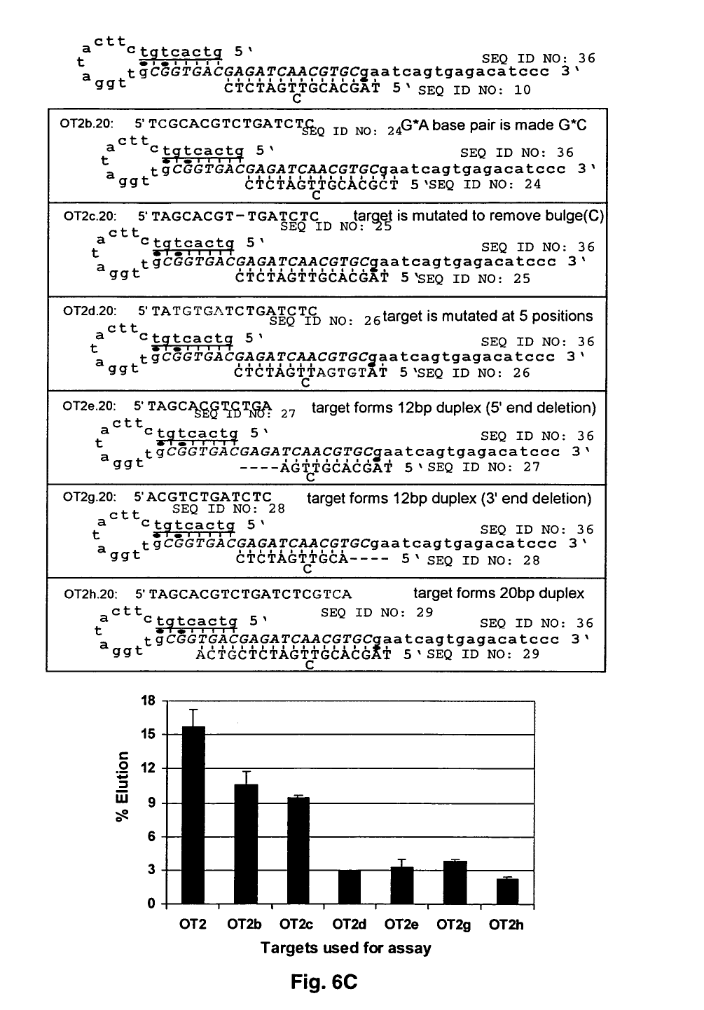

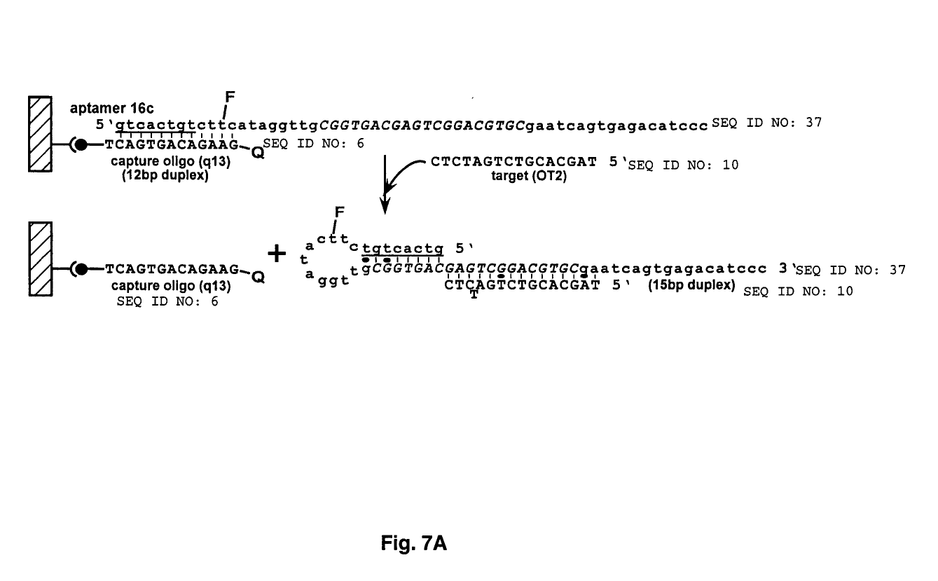

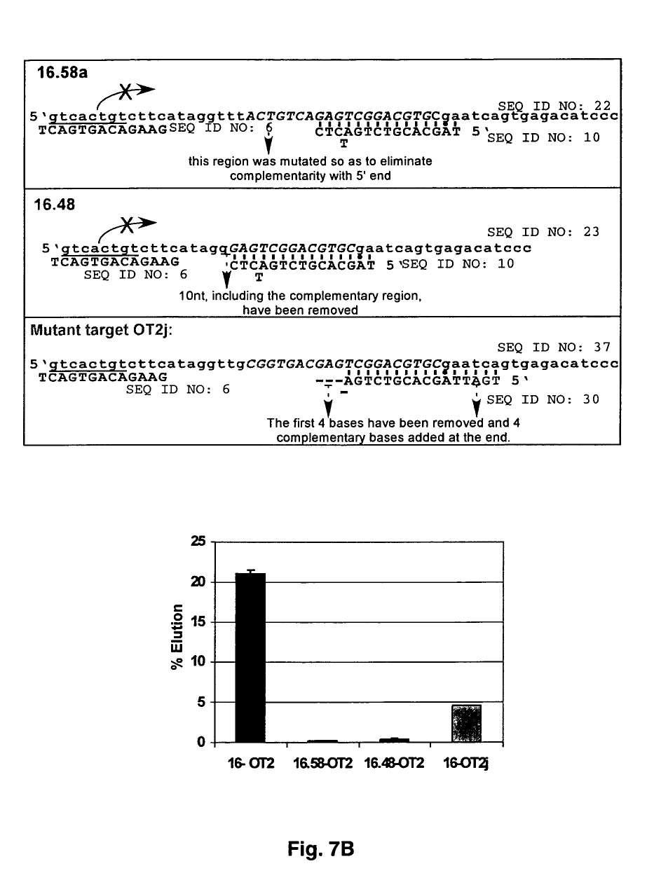

Beacon variants were designed based on the beacons 14a (FIG. 6A) and 16c (FIG. 7A). Binding assays with individual, selected beacons and designed variants were also performed as described above. The designed variants 14a.58, 14a.53a, 14a.53b, 14a.48, 14a.43, 14a.42, 16.58, and 16.48 were all assayed for their ability to be eluted by target OT2 (FIG. 6B, FIG. 7B). Beacon 14a was assayed for its ability to be eluted by targets OT2b.20, OT2c.20, OT2d.20, OT2e.20, OT2g.20 and OT2h.20 (FIG. 6C). Beacon 16c was assayed for its ability to be eluted by target OT2j.20 (FIG. 5B). Similarly, the designed beacons cOT1 and cOT3 were assayed for their ability to be eluted by their respective targets, OT1 and OT3 or OT3b (FIG. 7C).

When the octamer motif within beacon 14a (5′ GCGGTGAC) (SEQ ID NO: 35) was mutated to eliminate potential complementarity with the 5′ constant region to form variant 14a.58, the beacon could no longer be eluted by the target oligonucleotide (FIG. 6B). Similarly, deletion constructs 14a.53b, 14a.48, 14a.43 that partially or completely removed the octamer motif could no longer be eluted by the target oligonucleotide. In contrast, when five residues outside the octamer motif were deleted to form beacon variant 14a.53a, the elution characteristics of the beacon remained almost unchanged.

Mutant target oligonucleotides were also assayed for their ability to elute beacons (FIG. 6C). When the predicted complementarity of the target oligonucleotide was mutated either by changing five residues in tandem, target variant OT2d.20, or by deleting residues, target variants OT2e.20 and OTg.20, the extent of elution was decreased, further confirming that helix formation between the target oligonucleotide and the beacon is important for elution. Additionally, when the predicted complementarity of the target oligonucleotide was extended into the region required for the formation of the predicted hairpin stem, target variant OT2h.20, the extent of elution was decreased. These latter results are consistent with the postulated model, since any new base-pairs that are formed with the target oligonucleotide would have interfered with the formation of the stem-loop and stacked helical junction and thus with the dissociation of the selected beacon from the capture oligonucleotide.

Approximately 3% of the wild-type beacon was eluted with mutant target oligonucleotides while much lower levels of mutant beacons were eluted with the wild-type target oligonucleotide. These results are consistent with the finding that the wild-type beacon has a background elution rate of 3% (data not shown). Mutant beacons are less able to undergo the selected conformational change and thus should show a lower intrinsic elution rate irrespective of the nature of the target oligonucleotide.

While almost all results are consistent with the proposed structural hypothesis, a few substitutions do not fit the structure. Target oligonucleotide OT2b.20 contained an A to C substitution that should have replaced a postulated G:A base-pair in beacon 14a with a more stable G:C base-pair (FIG. 6C). Similarly, target oligonucleotide OT2c.20 was mutated to delete a predicted bulged base (FIG. 4C). In both instances, the fractions of eluted beacons unexpectedly decreased by slight amounts. These results indicate that a secondary or tertiary structure more complex than a simple Watson-Crick paired duplex may assist in the formation of the stacked helical junction.

Similar assays were also performed with beacon 16c to determine its mechanism of elution. When the octamer motif (5′ GCGGTGAC) (SEQ ID NO: 35) was again mutated, beacon variant 16.58a, or deleted, beacon variant 16.48, to eliminate complementarity the beacon no longer eluted in the presence of the target oligonucleotide (FIG. 5B), as previously observed with beacon variant 14a.58. When the target oligonucleotide was mutated to interfere with the formation of the stacked helical junction, as with target variant OT2j.20, elution again decreased markedly, further corroborating the suggested mechanism (FIG. 7B).

The basic mechanism for beacon elution enables the beacons to be modified more significantly and provides means to design new beacons. A minimal version of beacon 14a was designed in which the 3′ portion of the molecule, beyond the target binding domain, was removed to form variant 14a.42 (FIG. 6B). The beacon continued to show robust target-dependent elution.

Secondary structure of the aptamer beacons immobilized on the capture oligonucleotide may contribute to the mechanism of elution. For example, when the beacon is annealed to the capture oligonucleotide, the remaining non-paired portion of the beacon might fold to form a stem-loop structure that presents the oligonucleotide-binding site, much as a regular molecular beacon folds to present its oligonucleotide-binding site. If so, oligonucleotide-binding and thus elution might be facilitated. For the selected beacon 14a, it was possible that the sequences ttgCGGTG (SEQ ID NO: 62) and CGTCGga (SEQ ID NO: 63) that encompass the 5′ and 3′ends of the random insert, respectively, might pair with one another.

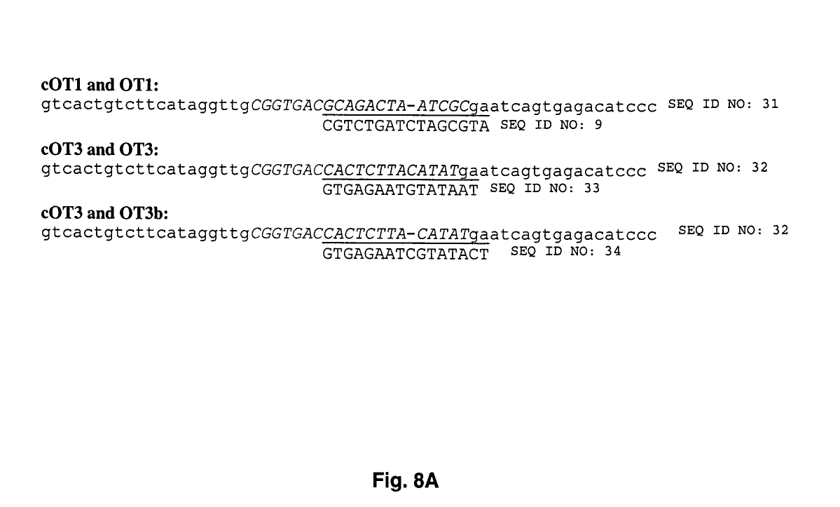

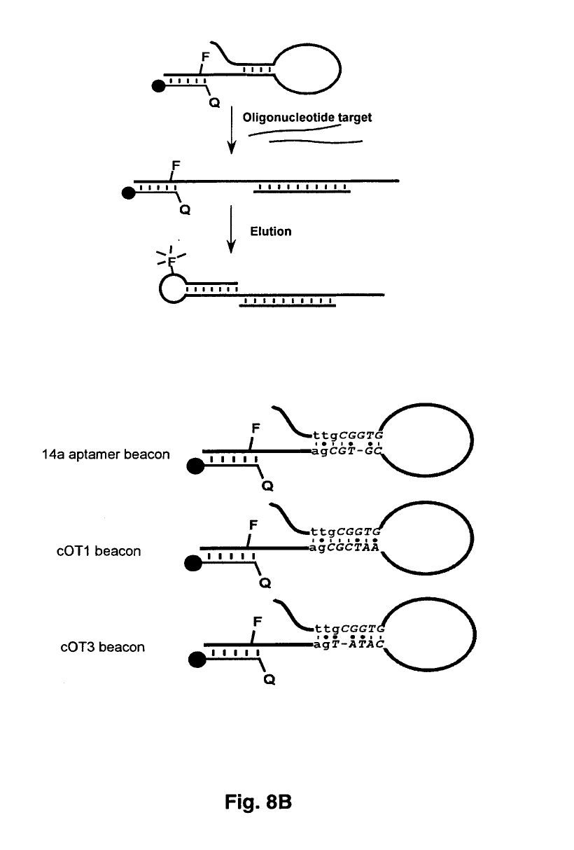

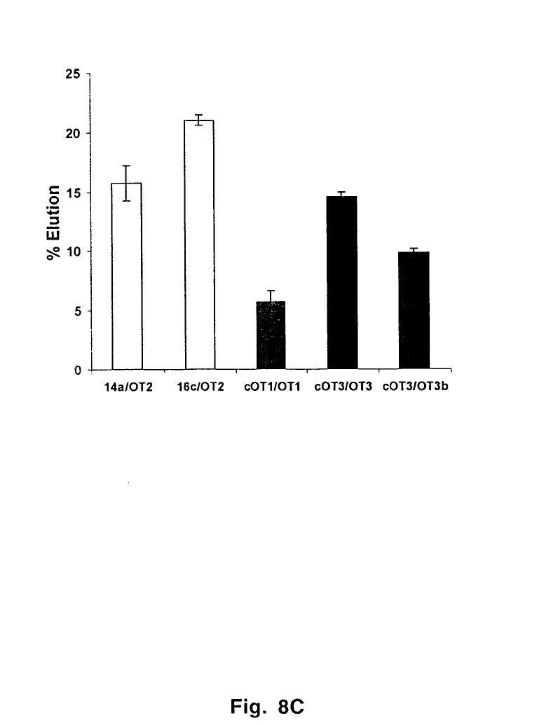

Two additional aptamer beacons, one that was complementary to target OT1, designated cOT1, and the other to a new and unrelated 16-mer sequence OT3, designated cOT3 or cOT3b were designed (FIG. 8A). Beacon cOT1 was designed to form a stronger hypothesized stem structure; beacon cOT3 was designed to not form a stem structure or to form an extremely weak structure (FIG. 8B). Both beacons showed oligonucleotide-specific elution which was greater than 4-fold above background elution, although they eluted less well than the original, selected beacons (FIG. 8C). A variant of target oligonucleotide OT3, designated OT3b, was also assayed and eluted cOT3 less well than target oligonucleotide OT3.

Similarly, a number of the originally selected molecular beacons should have formed the same hypothesized stem structure, but showed very different elution characteristics with the same oligonucleotide target, OT2; compare, for example, 16c and 24a. These results seem to indicate that the way in which an oligonucleotide binding site was presented was much less important for elution than the sequence of the oligonucleotide target and its binding site. This further emphasizes the generality and utility of the method. In addition, the selection appears to have led to the optimization of sequence and structural contributions to the elution mechanism. For example, the fact that the selected beacon that bound OT2 was eluted better than an equivalent designed beacon that bound OTI may explain why only OT2-dependent beacons were derived from the original selection.

### EXAMPLE 7

Binding Assays with Beacon Variants for N50 ssDNA

If the zinc binding aptamer beacons form an alternate stem formation upon binding zinc with concomitant release of the capture oligonucleotide, then the region 3′ of the common motif is expected to be dispensable for binding (FIGS. 9A-9B). Binding assays with individual, selected beacons and designed variants were also performed as described above. The variants Zn-36m1, Zn-36m2, Zn-36m3, Zn-36m4, Zn-36m5, Zn-36m6, and Zn-36m7 (FIGS. 9C-9D) and Zn-6m1, Zn-6m2, Zn-6m3, Zn-6m4, Zn-6m5 and Zn-6m6 (FIGS. 9F-9H) were designed such that the sequence between the predicted secondary structure motif and the 3′ constant region were removed. Zn-6, Zn-36 and the respective variants were all assayed for their ability to be eluted by Zn²⁺.

The elution characteristics of these minimized beacons, Zn-36m1 and Zn-6m1 (FIGS. 9E and 9I) were almost unchanged from the parent beacons, thus confirming the proposed model. In addition, in beacon Zn-6, a single adenosine is missing from the common pentamer motif, TAACT (SEQ ID NO: 129) becoming TACT (SEQ ID NO: 130). When a minimized beacon variant based on Zn-6 was designed to include this adenosine as well (Zn-6m2), it showed a higher affinity for zinc than the parent aptamer beacon (FIG. 9I).

In an attempt to locate the metal ion binding site(s), several other beacon variants were designed, again based on these 2 aptamer beacons and eluted with zinc (FIG. 9E). In beacon Zn-36m1, when the terminal loop, ttacg (SEQ ID NO: 131) was either replaced with a more stable GTGA loop (SEQ ID NO: 132) as in Zn-36m6, or removed along with the loop closing base pair, C.G as in Zn-36m2, the beacons could no longer be eluted by Zn²⁺ (FIG. 9E).

Similarly, when the two cytidines C28 and C29 which form a part of first bulge, were replaced with adenosines C28A and C29A as in Zn-36m4, again zinc binding was lost. In contrast, when the two cytidines were replaced with GT, C28G and C29T, as in Zn-36m3, such that this would lead to base pairing with the opposite AC bases and hence removal of bulge-1, some zinc dependent elution was retained. However, beacon variant Zn-36m3 could be eluted to the same extent even in the absence of zinc, suggesting that the higher elution observed might be due to a higher propensity of the variant to auto-elute since the alternate conformation is now more stabilized.

As a further confirmation of this explanation, it was observed that while greater than 90% of the beacons Zn-36, Zn-36m1, m2, m4 and m6 could be retained on the oligonucleotide affinity column following buffer washes, only 60% of the beacon Zn-36m3 was retained. In a similar manner, the single bulge cytidine residue, C33, was replaced with GA to remove the second bulge and replace it with a stable, continuous helix as in Zn-36m5. This time, only 45% of the beacon variant was retained following buffer washes and perhaps due to this reason, the beacon did not exhibit zinc-specific elution.

Assays were also performed with variants of beacon Zn-6m1 (FIG. 9H). As with beacon Zn-36m1, when the entire terminal loop I, ttacgGAG (SEQ ID NO: 133), as in Zn-6m3, or the part of the loop derived from the 5′ constant region, i.e., ttacg (SEQ ID NO: 131) or loop I′, as in Zn-6m6, was replaced with a GTGA loop (SEQ ID NO: 132), the variants could no longer be eluted by Zn²⁺. Beacon variant Zn-6m6 was tested to allow for the possibility of an alternate secondary structure, and hence terminal loop sequence, for aptamer beacon Zn-6m1 (FIG. 9F). Similarly, mutations which lead to the removal of the bulge regions, e.g., T31G and C32T in Zn-6m4 and C36GA in Zn-6m5, lead to poorer retention on the column (45-50% vs. 90%) and either no elution or zinc independent auto-elution.

However, no conclusive information about the zinc binding residues or sites could be obtained from these experiments. The terminal loop of the proposed secondary structure seems to be important for binding, but the sequence and the structural requirement of the two internal bulges could not be established. More than likely, the 9-10 bp duplex region involving the 5′ end of the aptamer beacons, stem-1, may not be involved in Zn²⁺ binding, since, barring a few non-conserved mismatches, the sequence and helical structure of this region is identical to that of the aptamer beacon-capture oligonucleotide hybrid. Thus, if Zn²⁺ were bound by stem-1, then all the sequences in the N50 pool would be expected to bind Zn²⁺ when still bound on the oligonucleotide affinity column and there would be no driving force for alternate secondary structure formation and elution.

To ascertain whether the aptamer beacons contain more than one independent Zn²⁺ binding site, the elution of beacon Zn-6m2 from the oligonucleotide affinity column in the presence of Zn²⁺ was assessed as a function of Zn²⁺ concentration (FIG. 9J). The binding data was monophasic and seem to indicate a single binding site for the Zn²⁺ ion.

To determine which residues in the minimized aptamer contribute to the specific recognition of Zn²⁺, a doped sequence population based on the predicted secondary structure of the highest affinity aptamer beacon, Zn-6m2, was prepared. For determination of the binding properties of Zn-6m2, 40 pmol of end-labeled aptamer was incubated with varying concentrations of Zn²⁺(0.2 mM to 2 mM), and the fraction eluting specifically with Zn²⁺ was estimated as done above for the different rounds of selection. Also, every round of the doped selection was assayed in a similar manner for its ability to be eluted by Zn²⁺.

Each position in the original random sequence region contained 55% wild type residues and 15% of each non wild type residue. The starting population should have contained all possible single to nine base substitutions. This level of doping should have been sufficient not only to ascertain the residues required for function, but also to explore the sequence space around the aptamer beacon for higher affinity species.

Six rounds of doped selection were performed at high stringency. The DNA to zinc ratio was increased to increase competition between the beacons and the incubation time for selection was decreased to select species with higher affinity and zinc-dependent elution. However, the doped selection did not progress as expected and the zinc-dependent elution ability was not found to improve over successive rounds. This might be due to the considerably higher stringency conditions applied for the doped selection; the incubation time with target was also reduced from 25 min to 8 min during the selection. In addition, the concentrations of zinc used were approximately 300-400 fold lower than the apparent K_{d} of the parent aptamer Zn-6m2. Since the doped library was derived from the aptamer beacon Zn-6m2, it is possible that the library may not contain any species with the expected higher affinity and hence no aptamers could be selected under the conditions used.

### EXAMPLE 8

Fluorescence Measurements

All fluorescence measurements were made on a PTI Quantamaster QM-4/2003SE spectrofluorimeter (Photon Technology International, Ontario, Canada). Beacons were generated for fluorescence measurements by NaOH mediated strand separation of fluorescently labeled DNA from its biotinylated complement immobilized on streptavidin-agarose column. The beacons at 50 nM final concentrations were annealed with capture oligonucleotide (100 nM) in a selection buffer (for oligo targets: 20 mM Tris, 7.5, 150 mM NaCl, 10 mM MgCl₂ or for Zn: 50 mM HEPES pH7.0, 300 mM NaCl, 0.5 mM MgCl₂) by heating to 94° C. for 30 sec and 45° C. for 90 sec and cooling to room temperature over 10 minutes. Background fluorescence was first measured by adding 480 μl of selection buffer to a fluorimeter cell. The beacon:capture oligonucleotide complexes (10 μl) were then added and fluorescence was monitored over time. Once a steady fluorescent signal had been achieved, either 10 μl of a heat-denatured solution of oligonucleotide target or 10 mL of selection buffer containing Zn²⁺ at various concentrations was added. The fluorescence response was monitored over 15 minutes by exciting the samples at 494 nm, the λₑₓ for fluorescein, and measuring the fluorescence intensity at 518 nm, the λₑₘ for fluorescein. The signal-to-background ratio was calculated as:
I=(Fₒₚₑₙ−F_{buffer})/(F_{closed}−F_{buffer}),
where for oligo targets Fₒₚₑₙ is the fluorescence of the beacon:capture complex, e.g., 14a or 16c hybridized to q13, in the presence of target, F_{closed} is the fluorescence of the beacon:capture complex in the absence of target or for zinc where Fₒₚₑₙ is the fluorescence of the beacon:capture oligonucleotide complex, e.g., Zn-6m2 hybridized to q12.50, in the presence of target, F_{closed} was the fluorescence of the beacon:capture oligonucleotide complex in the absence of target and F_{buffer} is the background fluorescence of the appropriate selection buffer solution alone.

The fluorescence quenching of the Zn-6m2 aptamer in the presence of zinc was monitored in a similar manner, except that this time the aptamer alone was heat denatured and the fluorescence response in the presence of Zn²⁺ was monitored over 4 minutes. The K_{d} value of the aptamer was estimated by curve fitting using the program Kaleidagraph (Synergy software, Reading, Pa.).

For the specificity measurements, the fluorescence response was represented as relative change in fluorescence to better represent the fluorescence change, i.e., increase versus quenching, in the presence of metal ions. The relative change in fluorescence, DRFU, was calculated as:
DRFU=(Fₓ−F₀)/F₀,
where the Fₓ was the fluorescence of the beacon:capture oligonucleotide complex in the presence of 2 mM target metal ion 4 min after addition, and F₀ was the fluorescence of the beacon:capture oligonucleotide complex in the absence of target.

The limits-of-detection for the selected molecular beacons were calculated by plotting I versus concentration at 15 minutes in the linear response range of 0 to 100 nM target oligonucleotide. Best-fit lines were calculated and the limits-of-detection were taken to be the concentration value at which I=3, a value previously used and described (12, 79).

For the doubly-labeled molecular beacons, the real time fluorescence response was monitored for both fluorescein and Texas Red (λₑₓ=595 nm and λₑₘ=615 nm) at each concentration of target oligonucleotide. The fluorescence response of each fluorphore was measured separately by exciting the beacon:capture oligonucleotide complex in the presence of various concentrations of target oligonucleotide at the excitation maxima of the fluorphore and recording the emission at the emission maxima. The value of I was evaluated after 15 minutes and the percent change in fluorescence was normalized according to the equation:
I=(I−Iₘᵢₙ)/(Iₘₐₓ−Iₘᵢₙ),
where I is the value at any particular target concentration, Iₘᵢₙ is the minimum value of I, i.e., the emission of fluorescein at 0 nM target and of Texas Red at 1000 nM target, and Iₘₐₓ is the maximum value of I, i.e., the emission of fluorescein at 1000 nM target and of Texas Red at 0 nM target.

The data set for fluorescein was fit to the equation:
Y=AX/(X+B),
where Y is the percent change in the fluorescence of fluorescein at a given target concentration, X is I, A is the increase in the fluorescence of fluorescein at saturating target concentrations, and B is the apparent dissociation constant value. Conversely, the data set for Texas red was fit to the equation:
Y=100+[AX/(X+B)],
where Y is the percent change in the fluorescence of Texas Red at a given target concentration, X is I, A is the decrease in the fluorescence of Texas Red at saturating target concentrations and B is the apparent dissociation constant value.

### EXAMPLE 9

Beacon Preparation for Fluorescence Measurements

Beacons were generated by reverse-transcribing RNA with Superscript II reverse transcriptase (Invitrogen, Carlsbad, Calif.) at 42° C. for 55 min. Template RNA was removed by ribonucleolytic degradation with several different ribonucleases. RNase A (Ambion, Austin, Tex.) and Ribonuclease H (Ambion, Austin, Tex.) first were added to the reverse transcription reaction and the mixture was incubated at 37° C. for 30 min, followed by 85° C. for 90 sec and 37° C. for 2 min. Next RNase I (Ambion, Austin, Tex.) and Riboshredder (Epicenter Technologies, Madison, Wis.) were added and the sample was incubated further at 37° C. for one hour. The single-stranded DNA was extracted with a mixture of phenol:chloroform and then chloroform alone, gel-purified on a 10% denaturing polyacryalmide gel, eluted overnight, and ethanol-precipitated.

### EXAMPLE 10

Oligonucleotide-Dependent Increases in Fluorescence of Selected Beacons

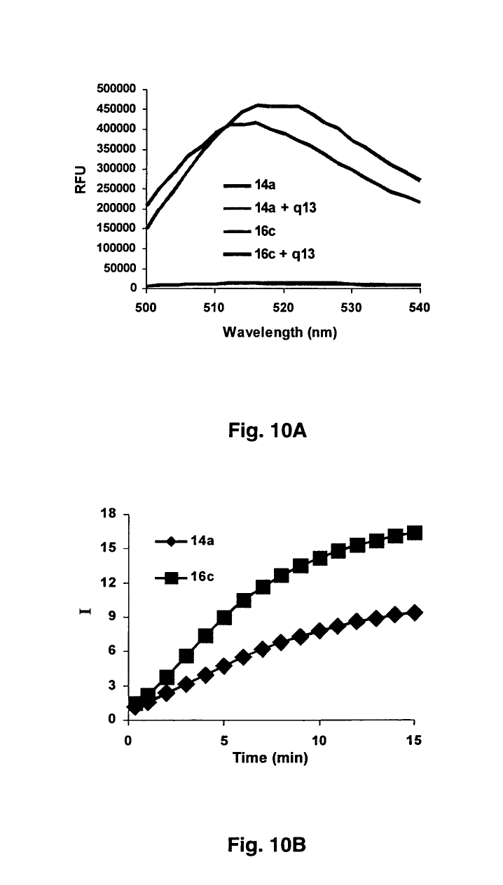

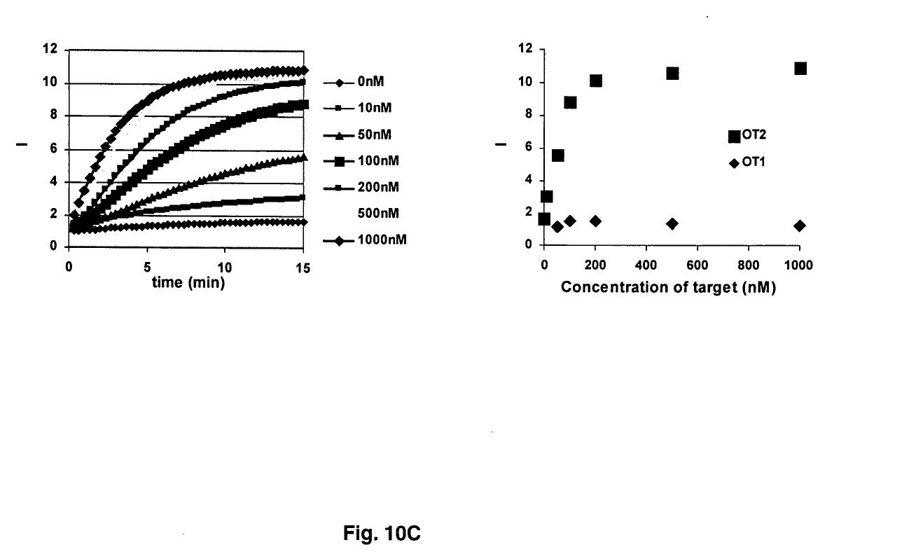

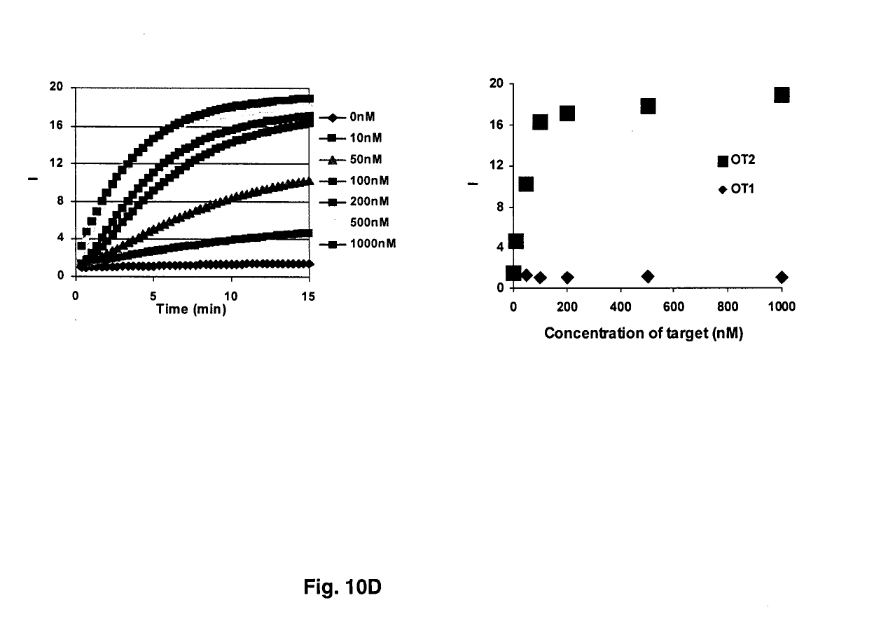

Changes in fluorescence upon the addition of target oligonucleotides were measured in order to determine if the eluted species could function like molecular beacons. Selected beacons bearing a fluorescein on T11 within the 5′ constant region were hybridized with a capture oligonucleotide containing a DABCYL moiety at its 5′ end. These positions were chosen to juxtapose the fluorescent reporter and the quencher, so that the fluorescent signal would be low in the absence of the target oligonucleotide. In fact, the hybridization of the two oligonucleotides resulted in up to 30-fold quenching for both beacons 14a and 16c (FIG. 10A). Addition of the oligonucleotide target (OT2) was predicted to facilitate the same conformational change that led to release from the column, and thus should displace the capture oligonucleotide bearing the quencher and also result in an increase in fluorescence intensity.

Beacon 14a showed a 9.5-fold increase in fluorescence in the presence of a two-fold molar excess of OT2, whereas beacon 16c showed a 16.5-fold increase in fluorescence (FIG. 10B). Both beacons 14a and 16c exhibited concentration-dependent increases in fluorescence. The selected molecular beacon 14a exhibited only 34% of its maximum possible fluorescence response, while beacon 16c exhibited an 85% response. The apparent K_{d} of beacon 14a for OT2 was 37±11 nM, and for beacon 16c a similar value was obtained, 34±8 nM.

The limit of target detection was approximately 14 nM for beacon 14a (FIG. 10C) and 3.6 nM for beacon 16c (FIG. 10D), values which are similar to the limits previously demonstrated for at least some designed molecular beacons (1,10,29, 80-82). The rate of fluorescence response increased with both target concentration and beacon concentration and appeared to follow second order kinetics. Based on the elution data, the selected beacons showed no fluorescence responses in the presence of the non-hybridizing target oligonucleotide OT1.

The relatively low responsivity of beacon 14a may be a consequence of the pool size used for selection. Designed molecular beacons (83) and other sequence sensors (84) previously disclosed form structures that can be readily disrupted by target oligonucleotides. The selected molecular beacons described herein disrupt a 12 base-pair, perfectly-paired duplex and instead are predicted to form an 8 base-pair stem-loop that contains two non-Watson-Crick pairings and a 15 base-pair duplex that contains at least one non-Watson-Crick pairing and frequently bulge residues as well (FIG. 3C).

The 23 base-pairs in the two stacked helices completely span the 20 nucleotide random sequence region and extend into the constant regions. It is contemplated that if a longer random sequence region was used a more stable, target-dependent conformer would be selected and the fluorescence response obtained would be greater. Nevertheless, even with the short pool that was employed at least one of the selected beacons, 16c, was able to obtain efficiencies similar to those seen for designed beacons. Beacon 16c may be much more efficient than beacon 14a because structures more complex than a Watson-Crick duplex ultimately may be responsible for the function of selected beacons.

Additionally, the selected beacons were generated by reverse transcription followed by RNase digestion of template RNA and optimal fluorescence response was found to be dependent on the purity of the samples. While the selected beacons always demonstrated a target-dependent increase in fluorescence, irrespective of how they were prepared, the magnitude of the fluorescence response decreased if the RNA template was incompletely degraded and removed. In order to optimize the responsivities of selected beacons, the cDNA produced by reverse transcription is treated sequentially with several different ribonucleases.

### EXAMPLE 11

Construction of Doubly-Labeled Molecular Beacons

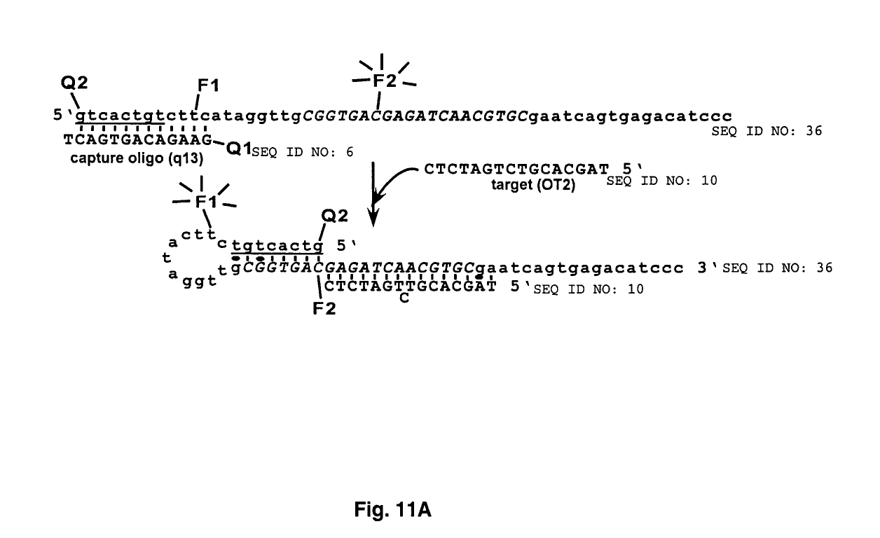

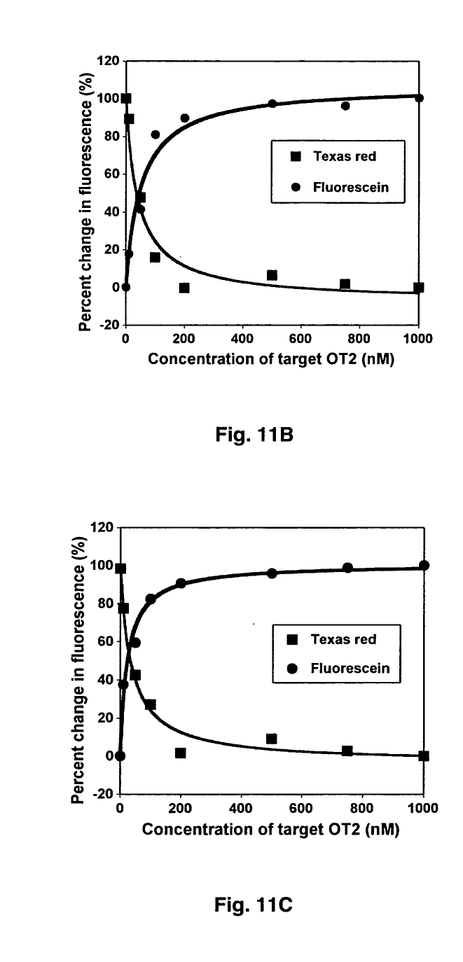

A second quencher, DABCYL, was introduced at the 5′ end of beacon constructs during chemical synthesis, and a second fluorescent reporter Texas Red was appended to a cytidine residue in the octamer motif at position 27 in the random region via post-synthetic chemical coupling. The two amine-modified beacon constructs, 14mb.58 and 16mb.58, were conjugated to the succinimidyl ester derivative of Texas Red-X (Molecular Probes, Eugene, Oreg.) (FIG. 11A). The coupling protocol provided with the dye was used with the exceptions that the oligonucleotide solution was heat-denatured at 75° C. for 3 minutes prior to setting up the reaction and the coupling reaction was carried out at a 4:1 dye:oligonucleotide ratio. After incubation for 12-16 hours at room temperature the labeled oligonucleotides were ethanol-precipitated two times and then gel-purified on a 12% denaturing polyacrylamide gel.

### EXAMPLE 12

Fluorescence Responsivities of Doubly-Labeled Molecular Beacons

The relative increase and decrease in these two fluorescent signals as a function of target concentration is shown for derivatives of beacon 14a (FIG. 11B) and beacon 16c (FIG. 11C). While it is possible that fluorescence resonance energy transfer can take place between fluorescein and Texas Red, the color-switching that was observed is more consistent with a dequenching of fluorescein followed by a subsequent quenching of Texas Red than with changes in fluorescence resonance energy transfer between the dyes themselves. The apparent K_{d} of the OT2:14a beacon complex was calculated based on fluorescein response to be 52±12 nM and on Texas Red response to be 43±11 nM. These values were slightly higher than those observed for the selected beacons with a single fluorphore, possibly due to the interference of the second dye or quencher with OT2 binding or the conformational transition. The response times for the color-switching beacons were on the order of 10 minutes, as previously observed for the selected beacons with a single fluorphore.

### EXAMPLE 13

Selected Beacons Show Zn²⁺-Dependent Increases in Fluorescence

To determine if the selected aptamers can function as beacons, their changes in fluorescence due to conformational changes on addition of Zn²⁺ were measured (FIG. 12A). The minimized aptamer beacons, Zn-6m2 and Zn-36m1, bearing a fluorescein moiety at an internal position, thymidine 11, were hybridized with the capture oligonucleotide q12.50 containing a DABCYL moiety at its 5′ end. As expected, the close proximity between fluorescein and DABCYL in the hybridized state led to quenching of fluorescence in the absence of target (FIGS. 12B-12C, curves I & II).

Addition of Zn²⁺ to the aptamer: quencher oligonucleotide complex in solution was predicted to facilitate the same conformational change that led to release from the column, and hence, result in an increase in fluorescence intensity. But interestingly, the addition of Zn²⁺ was observed to cause fluorescence quenching of the aptamer beacons (FIGS. 12B-12C, curves III and IV). The effect was more pronounced for beacon Zn-36m1; the fluorescence response of this aptamer beacon in complex with the quencher oligonucleotide could not be restored even in the presence of 2 mM Zn²⁺. Also, the emission wavelength of this beacon, both alone and in complex with quencher oligonucleotide, is being red shifted in the presence of Zn²⁺.

In contrast, on addition of 2 mM Zn²⁺, the fluorescence of beacon Zn-6m2 in complex with the quencher oligonucleotide was restored to the same extent (FIG. 12C, curve III) as the fluorescence of the aptamer beacon alone in the presence of the same concentration of Zn²⁺ (FIG. 12C, curve IV). This probably suggests that beacon Zn-6m2 is very efficient in binding Zn²⁺ and simultaneously releasing the quencher oligonucleotide, and the process probably goes to completion. Thus, in all likelihood, the optical response on Zn²⁺ binding must be due to the sequestration of zinc within the binding pocket.

In the case of Zn-36m1 however, the observed fluorescence response could not be completely rationalized. One possible explanation for the low fluorescence response of the aptamer: quencher oligonucleotide complex in the presence of Zn²⁺ is that beacon Zn-36m1 is not as efficient as Zn-6m2 in releasing the quencher oligonucleotide in the presence of Zn²⁺. However in radiolabeled assays where elution from a column was monitored, beacon Zn-36m1 showed a zinc-dependent elution behavior which was comparable to that of beacon Zn-6m2 (FIG. 5). Also, the aptamer alone was quenched ˜3 fold in the presence of Zn²⁺ and emission maximum was shifted by ˜12 nm.

Another possibility is that both the aptamer alone and the aptamer:quencher oligonucleotide complex may be binding Zn²⁺ within a specific binding pocket, and the fluorescein moiety is being quenched by Zn²⁺ upon binding. For example, the fluorescein moiety may either directly form a part of the binding pocket or be held in close proximity to Zn²⁺ upon binding. The bound Zn²⁺ is now at a higher effective concentration near the fluorphoree, and its electric field can perturb the fluorphore dipoles. The effect will be more pronounced in the excited state and hence, in a manner similar to the emission red shift caused by increase in solvent polarity, Zn²⁺ binding may be causing a shift in the emission curve of the fluorphoree. In addition, it has been observed before with other fluorescein derivatives that complex formation with transition metal cations leads to fluorescence quenching due to either an electron transfer or energy transfer between the metal ion and fluorphore (67,85). A more detailed study of the structure and mechanism of this beacon will need to be performed to fully understand its behavior.

Beacon Zn-6m2 in complex with quencher oligonucleotide exhibited target concentration-dependent increase in fluorescence (FIG. 13A). In the presence of 2 mM Zn²⁺, beacon Zn-6m2 showed a 5.5 fold increase in fluorescence. Also, unlike molecular beacons, the fluorescence response of the Zn²⁺-aptamer beacon was much faster and reached a stable level in less than 5 mins. In addition, the fluorescent aptamer alone showed target concentration-dependent decrease in fluorescence on addition of Zn²⁺(FIG. 13B). The fluorescence of the aptamer was quenched to 78% of its initial value in the presence of 2 mM Zn²⁺. The K_{d} of the aptamer was estimated from the quenching data to be 8.4±2.4 mM.

### EXAMPLE 14

Specific Fluorescence Response of the Selected Beacons

An important aspect for any metal ion sensor is its selectivity for the target ion. To determine the specificity of beacon Zn-6m2, its fluorescence response was monitored in the presence of 7 different metals (FIG. 14). At the same concentration, the fluorescence response for Zn²⁺ was at least 4.6 fold higher than the response for Mg²⁺ and Ca²⁺, the metal ions which are in greater abundance in biological media, and hence likely to cause interference in any sensor application for the beacon. The beacon was also selective for Zn²⁺, when compared to other transition metal ions like, Ni²⁺, Co²⁺, Mn²⁺and Fe²⁺, and showed fluorescence quenching in the presence of some of these metal ions.

This has been observed before with other macrocyclic Zn²⁺ sensors, such as Zinpyrs and ZnAFs, that fluorescence increases in the presence of zinc, but quenching occurs with other first row transition metal ions (39,67). However, Cd²⁺, which has the same d¹⁰ configuration as Zn²⁺ and occurs right below it in the periodic table, elicited a strong fluorescence response from the aptamer. This has been observed before with other zinc-sensors that responsivity is selective for both Zn²⁺ and Cd²⁺ (58,68).

The following references are cited herein:
1. Tyagi, et al., Nat. Biotechnol. 14, 303-8 (1996).
2. Nutiu et al., Nucleic Acids Res. 30, e94 (2002).
3. Marras, et al., Genet. Anal. 14, 151-6 (1999).
4. Marras, et al., Methods Mol Biol 212, 111-28 (2003).
5. Kostrikis, et al., Science 279, 1228-9 (1998).
6. Vet, et al., Proc Natl Acad Sci USA 96, 6394-9 (1999).
7. Piatek, et al., Nat Biotechnol 16, 359-63 (1998).
8. Tyagi, et al., Nat Biotechnol 16, 49-53 (1998).
9. Leone, et al., Nucleic Acids Res 26, 2150-5 (1998).
10. Sokol, et al., Proc Natl. Acad. Sci. USA 95, 11538-43 (1998).
11. Perlette, et al., Anal Chem 73, 5544-50 (2001).
12. Liu, et al., Anal Chem 71, 5054-9 (1999).
13. Liu, et al., Anal Biochem 283, 56-63 (2000).
14. Steemers, et al., Nat Biotechnol 18, 91-4 (2000).
15. Stojanovic, et al., Chembiochem. 2, 411-415 (2001).
16. Stojanovic, et al., J Am Chem Soc 124, 3555-3561 (2002).
17. Li, et al., Angew. Chem. Int. Ed. Engl. 39, 1049-52 (2000).
18. Fang, et al., Anal Chem 72, 3280-5 (2000).
19. Heyduk, et al., Nat Biotechnol 20, 171-6 (2002).
20. Hesselberth, et al., Rev. Mol. Biotechnol. 74, 15-25 (2000).
21. Jayasena, et al., Clin Chem 45, 1628-50 (1999).
22. Wilson, et al., Annu. Rev. Biochem 68, 611-47 (1999).
23. Rajendran, et al., Nucleic Acids for Reagentless Biosensors Optical Biosensors: Present and Future (Ligler, et al., Eds.), Elsevier Science B. V, Amsterdam 369-96 (2002).
24. Rajendran, et al., Comb. Chem. High Throughput Screen 5, 263-70 (2002).
25. Jhaveri, et al., J. Am. Chem. Soc. 122, 2469-73 (2000).
26. Li, et al., Biochem Biophys Res Commun 292, 31-40 (2002).
27. Stojanovic, et al., J. Am. Chem. Soc. 123, 4928-31 (2001).
28. Hamaguchi, et al., Anal Biochem 294, 126-31 (2001).
29. Yamamoto, et al., Genes Cells 5, 389-96 (2000).
30. Stojanovic, et al., J. Am. Chem. Soc. 122, 11547-48 (2000).
31. Nutiu, et al., J Am Chem Soc. 125, 4771-4778 (2003).
32. Jhaveri, et al., Nat Biotechnol. 18, 1293-7 (2000).
33. Pyle, A. M., J Biol Inorg Chem 2002, 7, (7-8), 679-90.
34. Tsien, R. Y., Am. J Physiol. 1992, 263, (4 Pt 1), C723-8.
35. Tsien, R. Y., In ACS Symposium Series (Fluorescent Chemosensors for Ion and Molecule Recognition), ed.; Czarnik, A. W., American Chemical Society: Washington, DC, 1993; Vol. 538, pp 130-146.
36. Miyawaki, et al., Nature 388, (6645), 882-7 (1997).
37. Allen, et al., Plant J 19, (6), 735-47 (1999)..
38. Chang, et al., Chem Biol 11, (2), 203-10 (2004).
39. Burdette, et al., J Am Chem Soc 123, (32), 7831-41 (2001).
40. Kimura, E. and Aoki, S., Biometals 14, (3-4), 191-204 (2001).
41. Thompson, et al., J Neurosci Methods, 118, (1), 63-75 (2002).
42. Walkup, et al., J. Am. Chem. Soc. 1997, 119, 3443-350 (1997).
43. Fierke, et al., Biometals 14(3-4), 205-22 (2001).
44. Ciesiolka, et al., Rna 1(5), 538-50 (1995).
45. Ciesiolka, et al., Rna 1996, 2, (8), 785-93.
46. Pan, et al., Nature, 358, (6387), 560-3 (1992).
47. Breaker, R. R. and Joyce, G. F., Chem Biol 1(4), 223-9 (1994).
48. Lehman, N. and Joyce, G. F., Nature 361(6408), 182-5 (1993).
49. Frank, D. N.and Pace, N. R., Proc Natl Acad Sci USA 94(26), 14355-60 (1997).
50. Cuenoud, et al., Nature, 375, (6532), 611-4 (1995).
51. Carmi, et al., Chem Biol 3, (12), 1039-46 (1996).
52. Carmi, et al., Bioorg Med Chem, 9(10), 2589-600 (2001).
53. Breaker, et al., Chem Biol 2(10), 655-60 (1995).
54. Li, et al. Nucleic Acids Res 28(2), 481-8 (2000).
55. Li, J. and Lu, Y., J Am Chem Soc 122, 10466-10467 (2000).
56. Liu, J. and Lu, Y., J Am Chem Soc 125(22), 6642-3 (2003).
57. Lu, et al., Biosens. Bioelectron. 18(5-6), 529-40 (2003).
58. Burdette, et al., J Am Chem Soc, 125(7), 1778-87 (2003).
59. apiero, H. and Tew, K. D., Biomed Pharmacother 57(9), 399-411 (2003).
60. Frederickson, et al., J Nutr 130, (5S Suppl), 1471S-83S (2000).
61. Frederickson, C. J. and Bush, A. I., Biometals, 14(3-4), 353-66 (2001).
62. Suh, et al., Brain Res 852, (2), 274-8 (2000).
63. Palmiter, et al., Proc Natl Acad Sci USA, 93(25), 14934-9 (1996).
64. Blencowe, D. K. and Morby, A. P., FEMS Microbiol Rev 27(2-3), 291-311 (2003).
65. Godwin, et al., J. Am. Chem. Soc. 118, 6514-6515 (1996).
66. Hirano, et al. Angew. Chem. Int. Ed. Engl. 39(6), 1052-1054 (2000).
67. Hirano, et al., J Am Chem Soc 124, (23), 6555-62 (2002).
68. Maruyama, et al., J Am Chem Soc 124, (36), 10650-1 (2002).
69. Green et al., Methods 2, 75-86 (1991).
70. Tyagi, et al., Nat Biotechnol 18, 1191-6 (2000).
71. Seeman, N. C. Nature 421, 427-431 (2003).
72. Yurke, et al., Nature, 406, 605-608 (2000).
73. Alberti, et al., Proc. Natl. Acad. Sci. U.S.A. 100, 1569-1573 (2003).
74. Robertson, et al., Nat. Biotechnol. 17(1), 62-6 (1999).
75. Famulok, M., Curr Opin Struct Biol 9(3), 324-9 (1999).
76. Hermann, T. and Patel, D. J., Science 287(5454), 820-5 (2000).
77. Cox, et al., Nucleic Acids Res., 30(20), E108-8 (2002).
78. Zuker, M., Nucleic Acids Res 31(13), 3406-15 (2003).
79. Zhang, et al., Angew. Chem. Int. Ed. Engl. 40, 402-05 (2001).
80. Nutiu, et al., Nucleic Acids Res. 30, e94 (2002).
81. Tan, et al., Chemistry 6, 1107-11 (2000).
82. Poddar, S. K. J. Virol. Methods 82, 19-26 (1999).
83. Tsourkas, et al., Nucleic Acids Res 31, 1319-30 (2003).
84. Li, et al., Nucleic Acids Res 30, E5 (2002).
85. de Silva, et al., Chem Rev 97(5), 1515-1566 (1997).
86. Robertson, et al., Nat. Biotechnol. 17, 62-6 (1999).
87. Cox, et al., Nucleic Acids Res. 30, E108-8 (2002).

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A method of selecting aptamer beacons in vitro comprising:
(a) generating a pool of single-stranded nucleic acid species, comprising:
a fluorphore F1; and
a random insert of N nucleotides;
(b) annealing said F1-labeled single-stranded nucleic acid species with a capture oligonucleotide comprising an F1 quenching moiety Q1 to form a capture pool;
(d) immobilizing said capture pool on a column;
(e) eluting said capture pool with at least one target;
(f) amplifying the F1-labeled single-stranded nucleic acid species comprising the eluate; and
(g) repeating steps (a) through (f) to select for the F1-labeled single-stranded nucleic acid species; wherein said selected 1-labeled single-stranded nucleic acid species comprise aptamer beacons.

2. The method of claim 1, wherein a 5′- and a 3′- region of said single-stranded nucleic acid species are constant regions.

3. The method of claim 2, wherein said fluorphore F1 is within the 5′-constant region.

4. The method of claim 1, wherein said capture oligonucleotide has a 3′- region sequence complementary to a 5′- region of said single-stranded nucleic acid species.

5. The method of claim 1, wherein said capture oligonucleotide further comprises biotin at the 3′-end.

6. The method of claim 1, further comprising:
cloning said selected F1-labeled single-stranded nucleic acid species.

7. The method of claim 1, further comprising:
increasing a molar ratio of pool F1-labeled single-stranded nucleic acid species to target(s) as steps (a) through (f) are repeated.

8. The method of claim 1, further comprising prior to step (e):
eluting said capture pool with an eluent suitable to remove immobilized nucleic acid species binding to non-targets; and
discarding the eluate.

9. The method of claim 8, wherein said non-target is one or more oligonucleotides having no sequence similarity with said target oligonucleotide and said eluent comprises a mixture of non-target oligonucleotides.

10. The method of claim 8, wherein said non-target is a metal ion and said eluent comprises a buffer with said non-target metal ion.

11. The method of claim 1, wherein F1 is fluorescein, Cascade blue, Alexa fluor 488 or Oregon green.

12. The method of claim 1, wherein Q1 is DABCYL or BHQ.

13. The method of claim 1, wherein the random insert of said F1-labeled ssDNA further comprises:
a fluorphore F2 different from fluorphore F1; and
an F2 quenching moiety Q2 on a 5′ end of said single-stranded nucleic acid species.

14. The method of claim 13, wherein F2 is Texas Red, rhodamine red or tamra.

15. The method of claim 13, wherein Q2 is DABCYL or BHQ.

16. The method of claim 1, further comprising:
sequencing said clones, wherein said clones having a motif of common residues at or near the 5′ end of the random insert comprise a family of aptamer beacons.

17. The method of claim 16, wherein said motif has the sequence shown in SEQ ID NO: 35.

18. The method of claim 16, wherein said family of aptamer beacons comprises at least one of the sequences shown in SEQ ID NOs.: 38-56.

19. The method of claim 16, wherein said motif has the sequence shown in SEQ ID NO: 129.

20. The method of claim 16, wherein said family of molecular beacons comprise at least one of the sequences shown in SEQ ID NOs.: 83-116.

21. The method of claim 1, wherein said random insert N is about 100 nucleotides or less.

22. The method of claim 21, wherein said random insert N is twenty nucleotides.

23. The method of claim 22, wherein said nucleic acid species has the sequence shown in SEQ ID NO: 1.

24. The method of claim 23, wherein said random insert N is fifty nucleotides.

25. The method of claim 1, wherein said nucleic acid species has the sequence shown in SEQ ID NO: 64.

26. The method of claim 1, wherein the capture oligonucleotide has the sequence shown in SEQ ID NO: 6 or in SEQ ID NO: 68.

27. The method of claim 1, wherein said target is an oligonucleotides, a metal ion, a peptide, a protein or a complex comprising a combination thereof.

28. The method of claim 27, wherein one of said target oligonucleotides has the sequence shown in SEQ ID NO: 10.

29. The method of claim 27, wherein said metal is Zn²⁺, Mn²⁺, Mg²⁺, Co²⁺, or Ni²⁺.

30. The method of claim 1, wherein said aptamer beacons have at least one of the sequences shown in SEQ ID NOS.: 38-56 or SEQ ID NOS: 83-116.

31. The method of claim 1, wherein said nucleic acid species is DNA, RNA, modified DNA or modified RNA.

32. Molecular beacons selected by the method of claim 1.

33. A method of detecting a ligand in solution, comprising the steps of:
a) determining an initial level of fluorescence of a fluorphore F1 attached within a 5′ region of the aptamer beacon of claim 19;
b) annealing said aptamer beacon with a capture oligonucleotide to form a captured beacon construct, said capture oligonucleotide comprising an F1 quenching moiety Q1, said quenching moiety quenching F1 upon binding;
c) immobilizing said captured beacon construct;
d) contacting said captured beacon construct with the solution;
e) interacting said ligand with said captured beacon whereby said captured beacon is released from said capture oligonucleotide; and
f) determining an increase in fluorescence of F1 from the quenched state of F1 upon the release of said captured beacon thereby detecting the ligand.

34. The method of claim 33, wherein said 5′ region of said aptamer beacon is a constant region.

35. The method of claim 34, wherein said fluorphore F1 is within the 5′-constant region.

36. The method of claim 33, wherein said capture oligonucleotide has a 3′ region sequence complementary to said 5′ region of said aptamer beacon.

37. The method of claim 33, wherein said capture oligonucleotide further comprises biotin at the 3′-end.

38. The method of claim 33, further comprising:
attaching a fluorphore F2 within a 5′ region of the random insert in said aptamer beacon, wherein said fluorphore F2 is different from fluorphore F1, each of said F1 and F2 exhibiting a distinct color upon fluorescing;
attaching an F2 quenching moiety Q2 on the 5′ end of the 5′ region of said aptamer beacon;
detecting the fluorescent color of F2 prior to step d;
quenching F2 with Q2 upon interacting said ligand with said captured beacon in step f; and
detecting a change in fluorescent color from F2 to F1 upon the release of said captured beacon.

39. The method of claim 38, wherein F2 is Texas Red, rhodamine red or tamra.

40. The method of claim 38, wherein said Q2 is DABCYL or BHQ.

41. The method of claim 33, wherein said aptamer beacon has a sequence shown in one of SEQ ID NOS.: 38-56 or SEQ ID NOS: 83-116.

42. The method of claim 33, wherein the capture oligonucleotide has the sequence shown in SEQ ID NO: 6 or in SEQ ID NO: 68.

43. The method of claim 33, wherein said ligand interacts with said captured beacon via binding thereto.

44. The method of claim 33, wherein said ligand is an oligonucleotide, a metal ion, a peptide, a protein or a complex comprising a combination thereof.

45. The method of claim 44, wherein said oligonucleotide ligand has the sequence shown in SEQ ID NO: 10.

46. The method of claim 44, wherein said metal is Zn²⁺, Mn²⁺, Mg²⁺, Co²⁺, or Ni²⁺.

47. The method of claim 33, wherein said fluorphore F1 is fluorescein, Cascade blue, Alexa fluor 488 or Oregon green.

48. The method of claim 33, wherein said F1 quenching moiety Q1 is DABCYL or BHQ.

49. A method of selecting a family of molecular beacons in vitro comprising the steps of:
(a) generating a pool of ssDNA having a random insert of N nucleotides between 5′ and 3′ constant regions, said ssDNA labeled with a fluorphore F1 in the 5′ constant region;
(b) annealing said F1-labeled ssDNA with a capture oligonucleotide complementary to the 5′ constant region of the F1-labeled ssDNA to form a capture pool, said oligonucleotide comprising:
a biotinylated 3′end; and
a 5′ end labeled with a fluorescence quenching moiety Q1, said quenching moiety Q1 proximate to F1 thereby quenching F1;
(d) immobilizing said capture pool on a column;
(e) eluting said capture pool with at least one target to release the F1-labeled ssDNA from said capture oligonucleotide, said F1-labeled ssDNA demonstrating an increase in fluorescence upon release from said capture oligonucleotide;
(f) collecting an eluate comprising an F1-labeled ssDNA pool bound to said target(s);
(g) amplifying the F1-labeled ssDNA comprising the eluate to form F1-labeled cDNA;
(h) repeating steps (a) through (g) to select for the F1-labeled ssDNA pool bound to said target;
(i) cloning said selected F1-labeled ssDNA, and
(j) sequencing said clones, wherein said clones having a motif comprising common residues at or near the 5′ end of the random insert comprise a family of molecular beacons.

50. The method of claim 49, further comprising:
increasing a molar ratio of pool F1-labeled ssDNA to target(s) as steps (a) through (g) are repeated.

51. The method of claim 49, further comprising prior to step (e):
eluting said capture pool with an eluent suitable to remove immobilized nucleic acid species binding to non-targets; and
discarding the eluate.

52. The method of claim 51, wherein said non-target is one or more oligonucleotides having no sequence similarity with said target oligonucleotide and said eluent comprises a mixture of non-target oligonucleotides.

53. The method of claim 5 1, wherein said non-target is a metal ion and said eluent comprises a buffer with said non-target metal ion.

54. The method of claim 49, wherein F1 is fluorescein, Cascade blue, Alexa fluor 488 or Oregon green.

55. The method of claim 49, wherein said random insert of said F1-labeled ssDNA further comprises:
a fluorphore F2 different from said fluorphore F1 within a 5′ end of said random insert such that said F2 fluoresces when the F1/F2-labeled ssDNA anneals with said capture oligonucleotide; and
a fluorescence quenching moiety Q2 on the 5′ end of the 5′ constant region; said quenching moiety Q2 proximate to F2 upon binding of said target ligand(s) to said F1/F2-labeled ssDNA thereby quenching F2.

56. The method of claim 55, wherein F2 is Texas Red, rhodamine red or tamra.

57. The method of claim 55, wherein Q2 is DABCYL or BHQ.

58. The method of claim 49, wherein said random insert N is about 100 nucleotides or less.

59. The method of claim 58, wherein said random insert N is twenty nucleotides.

60. The method of claim 59, wherein said nucleic acid species has the sequence shown in SEQ ID NO: 1.

61. The method of claim 49, wherein said random insert N is fifty nucleotides.

62. The method of claim 61, wherein said nucleic acid species has the sequence shown in SEQ ID NO: 64.

63. The method of claim 49, wherein the capture oligonucleotide has the sequence shown in SEQ ID NO: 6 or SEQ ID NO: 6.

64. The method of claim 49, wherein said target is an oligonucleotide, a metal ion, a peptide, a protein or a complex comprising a combination thereof.

65. The method of claim 64, wherein one of said target oligonucleotides has the sequence shown in SEQ ID NO: 10.

66. The method of claim 64, wherein said metal is Zn²⁺, Mn²⁺, Mg²⁺, Co²⁺, or Ni²⁺.

67. The method of claim 49, wherein said motif has the sequence shown in SEQ ID NO: 35.

68. The method of claim 49, wherein said family of aptamer beacons comprise at least one of the sequences shown in SEQ ID NOs.: 38-56.

69. The method of claim 49, wherein said motif has the sequence shown in SEQ ID NO: 129.

70. The method of claim 49, wherein said family of aptamer beacons comprise at least one of the sequences shown in SEQ ID NOs.: 83-116.

71. An aptamer beacon family selected by the method of claim 49.


SEQUENCE LISTING (133 sequences)

SEQ ID NO: 1 - DNA, 58 bp, Artificial Sequence
  modified_base (21)..(40): single-straded DNA sequence containing 20 randomized positions; n=a,c,g or t/u
gtcactgtcttcataggttgnnnnnnnnnnnnnnnnnnnngaatcagtgagacatccc

SEQ ID NO: 2 - DNA, 20 bp, Artificial Sequence
  Description of Artificial Sequence  20n.20 primer used to amplify DNA pool
gtcactgtcttcataggttg

SEQ ID NO: 3 - DNA, 38 bp, Artificial Sequence
  Description of Artificial Sequence  38.20 primer used to amplify DNA pool
ttctaatacgactcactatagggatgtctcactgattc

SEQ ID NO: 4 - DNA, 18 bp, Artificial Sequence
  Description of Artificial Sequence  primer contianing biotin at 5′ end
gggatgtctcactgattc

SEQ ID NO: 5 - DNA, 7 bp, Artificial Sequence
  Description of Artificial Sequence  7.oa20 capture oligonucleotide
agtgact

SEQ ID NO: 6 - DNA, 13 bp, Artificial Sequence
  Description of Artificial Sequence  13.oa20 capture oligonucleotide
gaagacagtgact

SEQ ID NO: 7 - DNA, 15 bp, Artificial Sequence
  Description of Artificial Sequence  15.oa20 capture oligonucleotide
tatgaagacagtgac

SEQ ID NO: 8 - DNA, 19 bp, Artificial Sequence
  Description of Artificial Sequence  19oa.20 capture oligonucleotide
caacctatgaagacagtga

SEQ ID NO: 9 - DNA, 16 bp, Artificial Sequence
  Description of Artificial Sequence  OT1.20 target oligonucleotide
atgcgatctagtctgc

SEQ ID NO: 10 - DNA, 16 bp, Artificial Sequence
  Description of Artificial Sequence  OT2.20 target oligonucleotide
tagcacgtctgatctc

SEQ ID NO: 11 - DNA, 18 bp, Artificial Sequence
  Description of Artificial Sequence RO1.18 oligonucleotide
gtagtgctccgtggattg

SEQ ID NO: 12 - DNA, 20 bp, Artificial Sequence
  Description of Artificial Sequence  RO2.20 oligonucleotide
tcgagggagagccatacctg

SEQ ID NO: 13 - DNA, 20 bp, Artificial Sequence
  Description of Artificial Sequence  RO3.20 oligonucleotide
tgcatgaggatgcaggatgc

SEQ ID NO: 14 - DNA, 20 bp, Artificial Sequence
  Description of Artificial Sequence  RO4.20 oligonucleotide
attgatgagtctgactgcct

SEQ ID NO: 15 - DNA, 19 bp, Artificial Sequence
  Description of Artificial Sequence  RO5.19 oligonucleotide
gcgactggacatcacgaga

SEQ ID NO: 16 - DNA, 58 bp, Artificial Sequence
  Description of Artificial Sequence  14a.58 oligonucleotide variant of 14a aptamer beacon
gtcactgtcttcataggtttactgtcagagatcaacgtgcgaatcagtgagacatccc

SEQ ID NO: 17 - DNA, 53 bp, Artificial Sequence
  Description of Artificial Sequence  14a.53a oligonucleotide variant of 14a aptamer beacon
gtcactgtcttcttgcggtgacgagatcaacgtgcgaatcagtgagacatccc

SEQ ID NO: 18 - DNA, 52 bp, Artificial Sequence
  Description of Artificial Sequence  14a.53b oligonucleotide variant of 14a aptamer beacon
tcactgtcttcataggttgcggagatcaacgtgcgaatcagtgagacatccc

SEQ ID NO: 19 - DNA, 48 bp, Artificial Sequence
  Description of Artificial Sequence  14a.48 oligonucleotide variant of 14a aptamer beacon
gtcactgtcttcatagggagatcaacgtgcgaatcagtgagacatccc

SEQ ID NO: 20 - DNA, 43 bp, Artificial Sequence
  Description of Artificial Sequence  14a.43 oligonucleotide variant of 14a aptamer beacon
gtcactgtcttcgagatcaacgtgcgaatcagtgagacatccc

SEQ ID NO: 21 - DNA, 42 bp, Artificial Sequence
  Description of Artificial Sequence  14a.42 oligonucleotide variant of 14a aptamer beacon
gtcactgtcttcataggttgcggtgacgagatcaacgtgcga

SEQ ID NO: 22 - DNA, 58 bp, Artificial Sequence
  Description of Artificial Sequence  16.58 oligonucleotide variant of 16c aptamer beacon
gtcactgtcttcataggtttactgtcagagtcggacgtgcgaatcagtgagacatccc

SEQ ID NO: 23 - DNA, 48 bp, Artificial Sequence
  Description of Artificial Sequence  16.48 oligonucleotide variant of 16c aptamer beacon
gtcactgtcttcatagggagtcggacgtgcgaatcagtgagacatccc

SEQ ID NO: 24 - DNA, 16 bp, Artificial Sequence
  Description of Artificial Sequence  OT2b.20 target oligonucleotide
tcgcacgtctgatctc

SEQ ID NO: 25 - DNA, 15 bp, Artificial Sequence
  Description of Artificial Sequence  OT2c.20 target oligonucleotide
tagcacgttgatctc

SEQ ID NO: 26 - DNA, 16 bp, Artificial Sequence
  Description of Artificial Sequence  OT2d.20 target oligonucleotide
tatgtgatctgatctc

SEQ ID NO: 27 - DNA, 12 bp, Artificial Sequence
  Description of Artificial Sequence  OT2e.20 target oligonucleotide
tagcacgtctga

SEQ ID NO: 28 - DNA, 12 bp, Artificial Sequence
  Description of Artificial Sequence  OT2g.20 target oligonucleotide
acgtctgatctc

SEQ ID NO: 29 - DNA, 20 bp, Artificial Sequence
  Description of Artificial Sequence  OT2h.20 target oligonucleotide
tagcacgtctgatctcgtca

SEQ ID NO: 30 - DNA, 16 bp, Artificial Sequence
  Description of Artificial Sequence  OT2j.20 target oligonucleotide
tgattagcacgtctga

SEQ ID NO: 31 - DNA, 57 bp, Artificial Sequence
  Description of Artificial Sequence cOT1 beacon complementary to oligonucleotide target OT1
gtcactgtcttcataggttgcggtgacgcagactaatcgcgatcagtgagacatccc

SEQ ID NO: 32 - DNA, 57 bp, Artificial Sequence
  Description of Artificial Sequence  cOT3 beacon complementary to 16-mer oligonucleotide target
gtcactgtcttcataggttgcggtgaccactcttacatatgatcagtgagacatccc

SEQ ID NO: 33 - DNA, 15 bp, Artificial Sequence
  Description of Artificial Sequence  OT3 target oligonucleotide
taatatgtaagagtg

SEQ ID NO: 34 - DNA, 16 bp, Artificial Sequence
  Description of Artificial Sequence  OT3b target oligonucleotide
tcatatgctaagagtg

SEQ ID NO: 35 - DNA, 8 bp, Artificial Sequence
  Description of Artificial Sequence  octamer motif within beacon 14a
gcggtgac

SEQ ID NO: 36 - DNA, 58 bp, Artificial Sequence
  Description of Artificial Sequence  molecular beacon
gtcactgtcttcataggttgcggtgacgagatcaacgtgcgaatcagtgagacatccc

SEQ ID NO: 37 - DNA, 58 bp, Artificial Sequence
  Description of Artificial Sequence  molecular beacon
gtcactgtcttcataggttgcggtgacgagtcggacgtgcgaatcagtgagacatccc

SEQ ID NO: 38 - DNA, 58 bp, Artificial Sequence; identical to SEQ ID NO: 36
  Description of Artificial Sequence  aptamer beacon 14a and 19a in family 1

SEQ ID NO: 39 - DNA, 58 bp, Artificial Sequence
  Description of Artificial Sequence  aptamer beacon 24a an 10a in family 1
gtcactgtcttcataggttgcggtgacgagatctgcgtgcgaatcagtgagacatccc

SEQ ID NO: 40 - DNA, 58 bp, Artificial Sequence
  Description of Artificial Sequence  aptamer beacon 30a in family 1
gtcactgtcttcataggttgcggtgacgagatcaagtgcggaatcagtgagacatccc

SEQ ID NO: 41 - DNA, 58 bp, Artificial Sequence
  Description of Artificial Sequence  aptamer beacon 3a in family 1
gtcactgtcttcataggttgcggtgacgagagaagacgtggaatcagtgagacatccc

SEQ ID NO: 42 - DNA, 58 bp, Artificial Sequence
  Description of Artificial Sequence  aptamer beacon 5a in family 1
gtcactgtcttcataggttgcggtgaccagacaagatcgcgaatcagtgagacatccc

SEQ ID NO: 43 - DNA, 58 bp, Artificial Sequence
  Description of Artificial Sequence  aptamer beacon 6a in family 1
gtcactgtcttcataggttgcggtgacgagacagacgtgcgaatcagtgagacatccc

SEQ ID NO: 44 - DNA, 59 bp, Artificial Sequence
  Description of Artificial Sequence  aptamer beacon 7a in family 1
gtcactgtcttcataggttgcggtgacgagayagacgkgcggaatcagtgagacatccc

SEQ ID NO: 45 - DNA, 58 bp, Artificial Sequence
  Description of Artificial Sequence  aptamer beacon 27a in family 1
gtcactgtcttcataggttgcggtgaccagaywaaattgcgaatcagtgagacatccc

SEQ ID NO: 46 - DNA, 58 bp, Artificial Sequence
  Description of Artificial Sequence  aptamer beacon 13b in family 1
gtcactgtcttcataggttgcggtgacgaatcagacgtgcgaatcagtgagacatccc

SEQ ID NO: 47 - DNA, 58 bp, Artificial Sequence
  Description of Artificial Sequence  aptamer beacon 25b in family 1
gtcaccgtcttcataggttgcggtgacgaatcagacgtgcgaatcagtgagacatccc

SEQ ID NO: 48 - DNA, 58 bp, Artificial Sequence; identical to SEQ ID NO: 37
  Description of Artificial Sequence  aptamer beacon 16c in family 1

SEQ ID NO: 49 - DNA, 58 bp, Artificial Sequence
  Description of Artificial Sequence  aptamer beacon 23c in family 1
gtcactgtcttcataggttgcggtgacgaggcggacgtgcgaatcagtgagacatccc

SEQ ID NO: 50 - DNA, 58 bp, Artificial Sequence
  Description of Artificial Sequence  aptamer beacon 28c in family 1
gtcactgtcttcataggttgcggtgacaratcggcgtgcggaatcagtgagacatccc

SEQ ID NO: 51 - DNA, 58 bp, Artificial Sequence
  Description of Artificial Sequence  aptamer beacon 1c in family 1
gtcactgtcttcataggttgcggtgacagatcagagtgcggaatcagtgagacatccc

SEQ ID NO: 52 - DNA, 58 bp, Artificial Sequence
  Description of Artificial Sequence  aptamer beacon lid in family 1
gtcactgtcttcataggttgggcggtgacagatcagacgcgaatcagtgagacatccc

SEQ ID NO: 53 - DNA, 59 bp, Artificial Sequence
  Description of Artificial Sequence  aptamer beacon 32d in family 1
gtcactgtcttcataggttgggcggtgaagacgggtaaggcgaatcagtgagacatccc

SEQ ID NO: 54 - DNA, 59 bp, Artificial Sequence
  Description of Artificial Sequence aptamer 8d in family 1
gtcactgtcttcataggttgggagtgacgagacagacgtgcgaatcagtgagacatccc

SEQ ID NO: 55 - DNA, 58 bp, Artificial Sequence
  Description of Artificial Sequence  aptamer beacon 29d in family 1
gtcactgtcttcataggttggagtgacgagttagacgtgcgaatcagtgagacatccc

SEQ ID NO: 56 - DNA, 58 bp, Artificial Sequence
  Description of Artificial Sequence aptamer beacon 15d in family 1
gtcactgtcttcataggttggtgacagatcaggtagtgcggaatcagtgagacatccc

SEQ ID NO: 57 - DNA, 58 bp, Artificial Sequence
  Description of Artificial Sequence  aptamer beacon 9 in family 1
gtcactgtcttcataggttgcacgggccctctagctcggggaatcagtgagacatccc

SEQ ID NO: 58 - DNA, 58 bp, Artificial Sequence
  Description of Artificial Sequence  aptamer beacon 12 in family 1
gtcactgtcttcataggttggggcaaatagtatgtaacccgaatcagtgagacatccc

SEQ ID NO: 59 - DNA, 58 bp, Artificial Sequence
  Description of Artificial Sequence  aptamer beacon 21 in family 1
gtcactgtcttcataggttgggcaggggcgtaggtcagccgaatcagtgagacatccc

SEQ ID NO: 60 - DNA, 59 bp, Artificial Sequence
  Description of Artificial Sequence  aptamer beacon 22 in family 1
gtcactgtcttcataggttgcagcggaccagactggagcgcgaatcagtgagacatccc

SEQ ID NO: 61 - DNA, 58 bp, Artificial Sequence
  Description of Artificial Sequence  aptamer beacon 31 in family 1
gtcactgtcttcataggttgggccgggtatgaagaaggccgaatcagtgagacatccc

SEQ ID NO: 62 - DNA, 8 bp, Artificial Sequence
  Description of Artificial Sequence  5′ end of random insert in aptamer beacon 14a
ttgcggtg

SEQ ID NO: 63 - DNA, 7 bp, Artificial Sequence
  Description of Artificial Sequence  3′ end of random insert in aptamer beacon 14a
cgtcgga

SEQ ID NO: 64 - DNA, 113 bp, Artificial Sequence
  Description of Artificial Sequence  DNA pool
gcatcagttagtcattacgcttacgnnnnnnnnnnnnnnnnnnnnnnnnnnnnnnnnnnnnnnnnnnnnnnnnnnattgtgaagtcgtgtccctatagtgagtcgtattagaa

SEQ ID NO: 65 - DNA, 25 bp, Artificial Sequence
  Description of Artificial Sequence  25.50 primer used to amplify DNA pool
gcatcagttagtcattacgcttacg

SEQ ID NO: 66 - DNA, 38 bp, Artificial Sequence
  Description of Artificial Sequence 38.50 prime used to amplify DNA pool
ttctaatacgactcactatagggacacgacttcacaat

SEQ ID NO: 67 - DNA, 18 bp, Artificial Sequence
  Description of Artificial Sequence  18.50 primer that contains biotin at its 5′ end
gggacacgacttcacaat

SEQ ID NO: 68 - DNA, 12 bp, Artificial Sequence
  Description of Artificial Sequence q12.50 capture oligonucleotide
actaactgatgc

SEQ ID NO: 69 - DNA, 93 bp, Artificial Sequence
  Description of Artificial Sequence  Zn-6ml oligonucleotide variant of Zn-6 aptamer beacon
gcatcagttagtcattacgcttacggcccgatcctaacttgcaaaggttcgtcgtcgcaatctgcgtggagggcgattgtgaagtcgtgtccc

SEQ ID NO: 70 - DNA, 60 bp, Artificial Sequence
  Description of Artificial Sequence  Zn-6m2 oligonucleotide variant of Zn-6 aptamer beacon
gcatcagttagtcattacgcttacggcccgatcctaacttgcattgtgaagtcgtgtccc

SEQ ID NO: 71 - DNA, 53 bp, Artificial Sequence
  Description of Artificial Sequence  Zn-6m3 oligonucleotide variant of Zn-6 aptamer beacon
gcatcagttagtcattacgcccgatcctaacttgcattgtgaagtcgtgtccc

SEQ ID NO: 72 - DNA, 60 bp, Artificial Sequence
  Description of Artificial Sequence  Zn-6m4 oligonucleotide variant of Zn-6 aptamer beacon
gcatcagttagtcattacgcttacggcgtgatcctaacttgcattgtgaagtcgtgtccc

SEQ ID NO: 73 - DNA, 60 bp, Artificial Sequence
  Description of Artificial Sequence  Zn-6m5 oligonucleotide variant of Zn-6 aptamer beacon
gcatcagttagtcattacgcttacggcaagatcctaacttgcattgtgaagtcgtgtccc

SEQ ID NO: 74 - DNA, 61 bp, Artificial Sequence
  Description of Artificial Sequence  Zn-6m6 oligonucleotide variant of Zn-6 aptamer beaco
gcatcagttagtcattacgcttacggcccgatgactaacttgcattgtgaagtcgtgtccc

SEQ ID NO: 75 - DNA, 59 bp, Artificial Sequence
  Description of Artificial Sequence Zn-36ml oligonucleotide variant of Zn-36 aptamer beacon
gcatcagttagtcattacgcgtgagcccgatcctaacttgcattgtgaagtcgtgtccc

SEQ ID NO: 76 - DNA, 64 bp, Artificial Sequence
  Description of Artificial Sequence  Zn-36m2 oligonucleotide variant of Zn-36 aptamer beacon
gcatcagttagtcattacgcttacggcggctctatcctactgatatattgtgaagtcgtgtccc

SEQ ID NO: 77 - DNA, 65 bp, Artificial Sequence
  Description of Artificial Sequence  Zn-36m3 oligonucleotide variant of Zn-36 aptamer beacon
gcatcagttagtcattacgcttacggcggctctatcctaactgatatattgtgaagtcgtgtccc

SEQ ID NO: 78 - DNA, 60 bp, Artificial Sequence
  Description of Artificial Sequence Zn-36m4 oligonucleotide variant of Zn-36 aptamer beacon
gcatcagttagtcattacgcgtgagctctatcctactgatatattgtgaagtcgtgtccc

SEQ ID NO: 79 - DNA, 64 bp, Artificial Sequence
  Description of Artificial Sequence  Zn-36m5 oligonucleotide variant of Zn-36 aptamer beacon
gcatcagttagtcattacgcttacggcggcgttatcctactgatatattgtgaagtcgtgtccc

SEQ ID NO: 80 - DNA, 65 bp, Artificial Sequence
  Description of Artificial Sequence Zn-36m3 oligonucleotide variant of Zn-36 aptamer beacon
gcatcagttagtcattacgcttacggcggctctatgactactgatatattgtgaagtcgtgtccc

SEQ ID NO: 81 - DNA, 63 bp, Artificial Sequence
  Description of Artificial Sequence  Zn-36m7 oligonucleotide variant of Zn-36 aptamer beacon
gcatcagttagtcattacgcgtgagcggctctatcctactgatatattgtgaagtcgtgtccc

SEQ ID NO: 82 - DNA, 65 bp, Artificial Sequence; identical to SEQ ID NO: 77
  Description of Artificial Sequence  doped sequence pool D22

SEQ ID NO: 83 - DNA, 75 bp, Artificial Sequence
  Description of Artificial Sequence  aptamer beacon Zn36 in family Zn1
gcatcagttagtcattacgcttacggcccgatcctaacttgcaaaggttcgtcgtcgcaatctgcgtggagggcg

SEQ ID NO: 84 - DNA, 75 bp, Artificial Sequence
  Description of Artificial Sequence  aptamer beacon Zn14 in family Zn1
gcatcagttagtcattacgcttacggcccgatcctaaccgaagaagaagaggttaataacgttccggacctggta

SEQ ID NO: 85 - DNA, 76 bp, Artificial Sequence
  Description of Artificial Sequence  aptamer beacon Zn13 in family Zn1
gcatcagttagtcattacgcttacggccggatcctaactggatgtttcaataggctcagacccgcaatacattcgc

SEQ ID NO: 86 - DNA, 74 bp, Artificial Sequence
  Description of Artificial Sequence  aptamer beacon Zn25 in family Zn1
gcatcagttatcattacgcttacggccgatcctaactgctttggatgagacattctgggacacgtgtcctgtgg

SEQ ID NO: 87 - DNA, 76 bp, Artificial Sequence
  Description of Artificial Sequence  aptamer beacon Zn16 in family Zn1
gcatcagttagtcattacgcttacggcggccgatcctaactcawgttggaatccgttaacacgtcgagctagtggg

SEQ ID NO: 88 - DNA, 73 bp, Artificial Sequence
  Description of Artificial Sequence  aptamer beacon Zn11 in family Zn1
gcatcagttagtcattacgcttacggcggccgatcctaacggtgctgttgtctctacgttctacaaggcatcc

SEQ ID NO: 89 - DNA, 76 bp, Artificial Sequence
  Description of Artificial Sequence  aptamer beacon Zn17 in family Zn1
gcatcagttagtcattacgcttacgggcggccgatcctaactgcggctaacacgttagcaaatgtcatctgattgc

SEQ ID NO: 90 - DNA, 75 bp, Artificial Sequence
  Description of Artificial Sequence  aptamer beacon Zn6 in family Zn1
gcatcagttagtcattacgcttacggcggctctatcctactgatattactgtccccttccgccagttgtgccgcg

SEQ ID NO: 91 - DNA, 75 bp, Artificial Sequence
  Description of Artificial Sequence  aptamer beacon Zn7 in family Zn1
gcatcagttagtcattacgcttacggcggccgtatcctaactaataatggagcggaaaacagcttcatacgcgtc

SEQ ID NO: 92 - DNA, 76 bp, Artificial Sequence
  Description of Artificial Sequence   aptamer beacon Zn35 in family Zn1
gcatcagttagtcattacgcttacggaggcagcatcctaactgtaactgtttaacgacttacaagacccagcgtcg

SEQ ID NO: 93 - DNA, 77 bp, Artificial Sequence
  Description of Artificial Sequence  aptamer beacon Zn21 in family Zn1
gcatcaggtagtcattaacgcttacggcggctgtatcctaatttgaagkggtaatggaaggaggttaaggacgatag

SEQ ID NO: 94 - DNA, 75 bp, Artificial Sequence
  Description of Artificial Sequence    aptamer beacon Zn10 in family Zn1
gcatcagttagtcattacgcttacggctcgtagcggccctatcctaacggtacgccaggtcgacagccagtgttc

SEQ ID NO: 95 - DNA, 75 bp, Artificial Sequence
  Description of Artificial Sequence  aptamer beacon Zn2 in family Zn1
gcatcagttagtcattacgcttacggcagcgtgcggctccatcctaactgcaagggccgctagtcacattgtgac

SEQ ID NO: 96 - DNA, 77 bp, Artificial Sequence
  Description of Artificial Sequence  aptamer beacon Zn33 in family Zn1
gcatcagttagtcattacgcttacggtgtgcgtatgcggcagatcctaacctgggacattcttccactagagactgc

SEQ ID NO: 97 - DNA, 75 bp, Artificial Sequence
  Description of Artificial Sequence  aptamer beacon Zn3 in family Zn1
gcatcagttagtcattacgcttacggcagcctgcctgtctacgagcggccgatcctaacgatcatagtcccgacg

SEQ ID NO: 98 - DNA, 75 bp, Artificial Sequence
  Description of Artificial Sequence  aptamer beacon Zn31 in family Zn1
gcatcagttagtcattacgcttacgggccattacggtggcacgagcggctcgatcctaatctgtgaaggaacgtc

SEQ ID NO: 99 - DNA, 75 bp, Artificial Sequence
  Description of Artificial Sequence  aptamer beacon Zn1 in family Zn1
gcatcagttagtcattacgcttacgggccatctacgactgggtaagaaagcggccgcatactagctgatttgttc

SEQ ID NO: 100 - DNA, 76 bp, Artificial Sequence
  Description of Artificial Sequence  aptamer beacon Zn8 in family Zn1
gcatcagttagtcattacgcttacgggcagcatgcacggagcggccgcatcttacgggtgtgtggcgggtagcact

SEQ ID NO: 101 - DNA, 75 bp, Artificial Sequence
  Description of Artificial Sequence  aptamer beacon Zn18 in family Zn1
gcatcagttagtcattacgcttacggcggccgtattaactgttggtatyaacaatcgtgctctgcgaagctcacg

SEQ ID NO: 102 - DNA, 74 bp, Artificial Sequence
  Description of Artificial Sequence aptamer beacon Zn30 in family Zn1
gcatcagttagtcattacgcttacggctaagcggctgttatcctaacatatgatgagttatcctcactagtacg

SEQ ID NO: 103 - DNA, 75 bp, Artificial Sequence
  Description of Artificial Sequence  aptamer beacon Zn34 in family Zn1
gcatcagttagtcattacgcttacggccggctgggtgtaatcctaacctgccctaactgctggggctggcgtgcc

SEQ ID NO: 104 - DNA, 75 bp, Artificial Sequence
  Description of Artificial Sequence  aptamer beacon Zn12 in family Zn2
gcatcagttagtcattacgcttacgttcgcggctaacttaggcgatatcaataagccacmatacgggcgatccgg

SEQ ID NO: 105 - DNA, 75 bp, Artificial Sequence
  Description of Artificial Sequence  aptamer beacon Zn28 in family Zn2
gcatcagttagtcattacgcttacgcagtccgtccgcgagtaactgaaaacctcaatggctcgacggtcacccgt

SEQ ID NO: 106 - DNA, 76 bp, Artificial Sequence
  Description of Artificial Sequence aptamer beacon Zn5 in family Zn2
gcatcagttagtcattacgcttacggcacgaagcgttcgcggctatctgaacgttggttcmcggatgtcttcactg

SEQ ID NO: 107 - DNA, 75 bp, Artificial Sequence
  Description of Artificial Sequence  aptamer beacon Zn9 in family Zn2
gcatcagttagtcattacgcttacgcccagcgaagagttcgcgagtaactgtgtcgattacaattccgattggac

SEQ ID NO: 108 - DNA, 76 bp, Artificial Sequence
  Description of Artificial Sequence  aptamer beacon Zn4 in family Zn2
gcatcagttagtcattacgcttacggagtgcccacgttgtgagcgtttggcggctagctggggatgtgtgtattcg

SEQ ID NO: 109 - DNA, 76 bp, Artificial Sequence
  Description of Artificial Sequence aptamer beacon Zn20 in family Zn2
gcatcagttagtcattaagcttacggtgggcaaaatgcagtgstgttagaaggttgcgagttactgcagatctggg

SEQ ID NO: 110 - DNA, 77 bp, Artificial Sequence
  Description of Artificial Sequence  aptamer beacon Zn15 in family Zn2
gcatcagttagtcattacgcttacggacggcaagtygtgcaaagcgtttgcggcttaacttaatgaattaaacgcgc

SEQ ID NO: 111 - DNA, 75 bp, Artificial Sequence
  Description of Artificial Sequence  aptamer beacon Zn26 in family Zn2
gcatcagttagtcattacgcttacgggtaccgttaggccaccttggttgagttaactgactaggctcaagacatc

SEQ ID NO: 112 - DNA, 75 bp, Artificial Sequence
  Description of Artificial Sequence  aptamer beacon Zn24 in family Zn2
gcatcagttagtcattacgcttacgttcgcagatctaactgagagcattaaggaatatcacgggctcgctcagtc

SEQ ID NO: 113 - DNA, 75 bp, Artificial Sequence
  Description of Artificial Sequence  aptamer beacon Zn19 in family Zn2
gcatcagttagtcattacgcttacgggccatggccggtaggtggttgcgactacggtgcagttcgtctatggaac

SEQ ID NO: 114 - DNA, 76 bp, Artificial Sequence
  Description of Artificial Sequence  aptamer beacon Zn22 in family Zn2
gcatcagttagtcattacgcttacgtaaggacgctgtctctgagtggtgttaccggcggctacaaattcgtctggt

SEQ ID NO: 115 - DNA, 75 bp, Artificial Sequence
  Description of Artificial Sequence  aptamer beacon Zn23 in family Zn2
gcatcagttagtcattaagcttacgggtgggcctgcacggtccttcaagtaagaggccgattcctactgcggtac

SEQ ID NO: 116 - DNA, 75 bp, Artificial Sequence
  Description of Artificial Sequence  aptamer beacon Zn32 in family Zn2
gcatcagttagtcattacgcttacggcgtgggtcgccagttgcgttcgcgactacggaggccacgttacaatccc

SEQ ID NO: 117 - DNA, 42 bp, Artificial Sequence
  Description of Artificial Sequence  aptamer beacon Zn36
gcatcagttagtcattacgcttacggcccgatcctaacttgc

SEQ ID NO: 118 - DNA, 42 bp, Artificial Sequence
  Description of Artificial Sequence  aptamer beacon Zn14
gcatcagttagtcattacgcttacggcccgatcctaaccgaa

SEQ ID NO: 119 - DNA, 42 bp, Artificial Sequence
  Description of Artificial Sequence  aptamer beacon Zn13
gcatcagttagtcattacgcttacggccggatcctaactgga

SEQ ID NO: 120 - DNA, 41 bp, Artificial Sequence
  Description of Artificial Sequence aptamer beacon Zn25
gcatcagttagtcattacgcttacggcggatcctaactgct

SEQ ID NO: 121 - DNA, 45 bp, Artificial Sequence
  Description of Artificial Sequence  aptamer beacon Zn16
gcatcagttagtcattacgcttacggcggccgatcctaactcatg

SEQ ID NO: 122 - DNA, 45 bp, Artificial Sequence
  Description of Artificial Sequence  aptamer beacon Zn11
gcatcagttagtcattacgcttacggcggccgatcctaacggtgc

SEQ ID NO: 123 - DNA, 47 bp, Artificial Sequence
  Description of Artificial Sequence  aptamer beacon Zn17
gcatcagttagtcattacgcttacgggcggccgatcctaactgcggc

SEQ ID NO: 124 - DNA, 46 bp, Artificial Sequence
  Description of Artificial Sequence  aptamer beacon Zn6
gcatcagttagtcattacgcttacggcggctctatcctactgatat

SEQ ID NO: 125 - DNA, 53 bp, Artificial Sequence
  Description of Artificial Sequence  aptamer beacon Zn10
gcatcagttagtcattacgcttacggctcgtagcggccctatcctaacggtac

SEQ ID NO: 126 - DNA, 53 bp, Artificial Sequence
  Description of Artificial Sequence  aptamer beacon Zn2
gcatcagttagtcattacgcttacggcagcgtgcggctccatcctaactgcaa

SEQ ID NO: 127 - DNA, 54 bp, Artificial Sequence
  Description of Artificial Sequence  aptamer beacon Zn33
gcatcagttagtcattacgcttacggtgtgcgtatgcggcagatcctaacctgg

SEQ ID NO: 128 - DNA, 63 bp, Artificial Sequence
  Description of Artificial Sequence  aptamer beacon Zn3
gcatcagttagtcattacgcttacggcagcctgcctgtctacgagcggccgatcctaacctgg

SEQ ID NO: 129 - DNA, 5 bp, Artificial Sequence
  Description of Artificial Sequence  pentamer motif
taact

SEQ ID NO: 130 - DNA, 4 bp, Artificial Sequence
  Description of Artificial Sequence  pentamer motif missing a single adenosine
tact

SEQ ID NO: 131 - DNA, 5 bp, Artificial Sequence
  Description of Artificial Sequence  terminal loop of aptamer beacon Zn-36m1
ttacg

SEQ ID NO: 132 - DNA, 4 bp, Artificial Sequence
  Description of Artificial Sequence  terminal loop of aptamer beacon Zn-36m6
gtga

SEQ ID NO: 133 - DNA, 8 bp, Artificial Sequence
  Description of Artificial Sequence  terminal loop of aptamer beacon Zn-6m1
ttacggag